(19) United States
(12) Patent Application Publication
(10) Pub. No.: US 2011/0065762 A1
(43) Pub. Date: Mar. 17, 2011

(54) METHODS OF USE OF ANTIVIRAL COMPOUNDS

(76) Inventors: Jizhou Wang, Eagleville, PA (US); Xiaodong Fan, Center Valley, PA (US); Lidia Cristian, Princeton Junction, NJ (US)

(21) Appl. No.: 12/881,056

(22) Filed: Sep. 13, 2010

Related U.S. Application Data
(60) Provisional application No. 61/241,659, filed on Sep. 11, 2009.

(51) Int. Cl. A61K 31/423; C07C 211/19; C07D 233/60; C07C 255/47; C07C 251/20; C07C 251/18; C07C 23/38; C07D 263/52; A61K 31/13; A61K 31/4164; A61K 31/155; A61K 31/04; A61P 31/16

(52) U.S. Cl. 514/375; 564/456; 564/459; 548/341.1; 558/429; 564/270; 564/225; 570/187; 548/223; 514/659; 514/662; 514/399; 514/638; 514/631; 514/740

(57) ABSTRACT

The present invention relates, in part, to methods of treatment, prevention, and inhibition of viral disorders. In one aspect, the present invention relates to inhibition of the M2 proton channel of influenza viruses (e.g. influenza A virus) and other similar viroporins (e.g., VP24 of Ebola and Marburg viruses; and NS3 protein of Bluetongue). The present invention further relates, inter alia, to compounds which have been shown to possess antiviral activity, in particular, inhibiting the M2 proton channel of influenza viruses.

### CROSS-REFERENCE TO RELATED APPLICATION

The present application claims priority to U.S. Provisional Application No. 61/241,659, filed Sep. 11, 2009, which is hereby incorporated by reference in its entirety.

### TECHNICAL FIELD

The present invention relates, in part, to methods of treatment, prevention, and inhibition of viral disorders. In one aspect, the present invention relates to inhibition of the M2 proton channel of influenza viruses (e.g., influenza A virus and/or influenza B virus) and other similar viroporins (e.g., VP24 of Ebola and Marburg viruses; and NS3 protein of Bluetongue). The present invention further relates to compounds which have been shown to possess antiviral activity, in particular, inhibiting the M2 proton channel (e.g., wild type and/or drug resistant influenza such as S31N influenza or other drug-resistant influenza strains) of influenza viruses and other similar viroporins.

### BACKGROUND

Viroporins are a growing class of membrane proteins that are important for viral replication and packaging. These proteins also affect cellular functions, including the cell vesicle system, glycoprotein trafficking and membrane permeability (Gonzalez et al., FEBS Lett., 2003, 552, 28-34). The M2 proton channel is a prototype for this class of proteins that is essential to the survival of the virus (Lamb et al., Wimmer E, editor, Receptor-Mediated Virus Entry into Cells, Cold Spring Harbor, N.Y., Cold Spring Harbor Press, 1994, p. 303-321).

Viroporins are essential components of a variety of viruses including Ebola, Marburg, Bluetongue, African horse sickness, foot and mouth disease, and Japanese encephalitis viruses. In particular, Ebola and Marburg viruses pose a particularly serious threat to human health and are classified as category A biowarfare agents by the Center for Disease Control (CDC) (Khan et al., MMWR, 2000, 49, RR-4, 1-14. VP24 from Ebola and Marburg viruses is an integral membrane protein that possesses viroporin activity similar to the M2 protein (Han et al., J. Virology, 2003, 77(3), 793-800). NS3 protein of Bluetongue is a viroporin that is critical for virus release (Han et al., J. Biol. Chem., 2004, 279, 41, 43092-43097). In addition, picronaviruses (Gonzalez et al., FEBS Lett., 2003, 552, 28-34), African horse sickness, and Japanese encephalitis encode proteins with viroporin activity that play central roles in viral pathogenesis (Van Niekerk et al., Virology, 2001, 279, 499-508; Chang et al., J. Virol., 1999, 73(8), 6257-6264).

Influenza viruses infect the upper and lower respiratory tracts and cause substantial morbidity and mortality annually. Influenza A viruses, which also infect a wide number of avian and mammalian species, pose a considerable public health burden with epidemic and pandemic potential. Influenza together with complications of the virus is consistently among the top 10 common causes of death, ranking higher than some other much more widely publicized killers, such as the HIV virus that causes AIDS. It is estimated that in annual influenza epidemics, 5-15% of the world's population contracts influenza, resulting in an estimated 3-5 million cases of severe illness and 250,000 to 500,000 deaths around the world from influenza-associated complications. In the U.S., 10%-20% of the population is infected with the flu every year, with an average 0.1% mortality. The flu causes 36,000 deaths each year in the U.S., and 114,000 hospitalizations. The cost of influenza epidemics to the U.S. economy is estimated at $3-15 billion. Approximately 20% to 40% of the world's population became ill during the catastrophic “Spanish” flu pandemic in 1918, which killed an estimated 40 to 50 million people worldwide and 675,000 people in the United States. The “Asian” flu pandemic of 1957 resulted in the deaths of approximately 69,800 people in the United States and 2.0 to 7.4 million worldwide. The H1N1 swine flu pandemic in 2009 has caused about 3,000 deaths worldwide to date.

Tamiflu (oseltamivir), which targets neuraminidase protein, is the only remaining orally administered anti-flu drug on the market and resistance to the drug is increasing with oseltamivir-resistant viruses arising during clinical use of the drug in children (Kiso et al., Lancet, 2004, 364, 759-65). Oseltamivir has been used for treatment of infected individuals and although it is FDA-approved for prophylaxis its usefulness for prophylactic treatment has been questioned in a recent systematic analysis of data from 51 controlled trials (Jefferson et al., Lancet, 2006, 367, 303-13). Thus, there is an immediate need to develop additional agents that inhibit the M2 proton channel and its drug-resistant forms, and in particular the most prevalent mutant form, S31N, but also in others including L26, V27, A30, and G34.

Influenza A and B viruses each encode a small oligomeric integral membrane protein, M2 of influenza A virus and BM2 of influenza B virus, each of which is a proton-selective ion channel. The M2 protein plays an important role during the early and late stages of the viral life cycle. Early in the cycle, the virus enters cells by receptor-mediated endocytosis, which places the virus into endosomal vesicles. Proton-pumping ATP-ases in the endosomal membrane lower the internal pH, which triggers the fusion of the viral envelope with the endosomal membrane and the release of the viral RNA into the cytoplasm. However, unless the inside of the virus is acidified prior to fusion, the RNA remains encapsulated by a matrix protein known as M1 (Ito et al., J. Virol., 1981, 65, 5491-8). The M2 protein provides a conduit for passage of protons into the interior of the virus, thereby promoting the dissociation of RNA from its matrix protein. This is a crucial step in uncoating of the virus and exposing its content to the cytoplasm of the host cell. In some strains of influenza A virus, the M2 protein is also important for equilibrating the pH of the lumen of the Golgi apparatus with the cytoplasm, thus preventing a premature conformational change in the viral hemagglutinin at the wrong time and in the wrong place (Ciampor et al., Acta Virologica, 1995, 39, 171-181). Inhibition of M2 at this later stage of the viral life cycle prevents viral maturation and release from the host cell.

Several features make M2 an excellent target for an anti-influenza drug. It is essential and present in all known isolates of influenza A virus, and it is already validated as a drug target. Although a variety of mutations occur naturally and can be isolated in cell culture, one mutant in particular, S31N, predominates in more than 98% of the transmissible resistant viral strains isolated from patients in the last decade (Bright et al., Lancet, 2005, 366, 1175-1181).

Thus, there is a great need for additional compositions and methods of treatment based on the use of antiviral compounds against key viral pathogens and, optionally, less prone to the development of resistance by those pathogens. Moreover, there is a great need for additional compositions and methods of treatment based on the use of antiviral compounds that are effective in the treatment of viral pathogens that have already developed resistance to existing antiviral agents. In particular, there is a great need for effective compositions and methods for the treatment of viral infections such as influenza, Ebola, Marburg, bluetongue, foot and mouth disease, African horse sickness, and Japanese encephalitis (including the strains that have already developed resistance to existing antiviral agents). The present invention is directed to these and other important ends.

### SUMMARY

The present invention provides, in part, methods for treating an influenza virus-affected disease state or infection comprising the step of administering to a subject in need thereof a composition comprising a compound of Formula I:

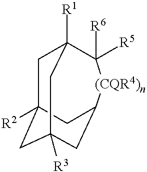

or a pharmaceutically acceptable salt thereof, wherein R¹, R², R³, R⁴, R⁵, and R⁶ are as defined herein.

In some embodiments, the present invention provides methods for treating a viral infection, such as influenza (e.g., wild-type influenza, such as wild-type influenza A or B, or one or more mutant varieties of influenza such as S31N influenza), Ebola, Marburg, bluetongue, foot and mouth disease, African horse sickness, and Japanese encephalitis, in a patient (including a human or an animal) comprising administering to a subject in need thereof a composition comprising a compound of Formula I as defined herein.

Also provided are compositions comprising a compound according to Formula I or a pharmaceutically acceptable salt or stereoisomer thereof, and a pharmaceutically acceptable carrier, diluent, or excipient.

Also disclosed are compounds according to Formula II

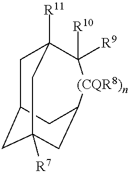

or a pharmaceutically acceptable salt thereof, wherein R⁷, R⁸, R⁹, R¹⁰, and R¹¹ are as defined herein, and compositions comprising one or more of such compounds or salts thereof, that can be administered for the treatment or prevention of a viral infection such as influenza (e.g., drug resistant influenza such as S31N influenza), Ebola, Marburg, bluetongue, foot and mouth disease, African horse sickness, and Japanese encephalitis in patients (including humans or other animals).

### DETAILED DESCRIPTION

The present invention may be understood more readily by reference to the following detailed description taken in connection with the accompanying figures and examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

The disclosures of each patent, patent application, and publication cited or described in this document are hereby incorporated herein by reference, in their entirety.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. Furthermore, when indicating that a certain chemical moiety “may be” X, Y, or Z, it is not intended by such usage to exclude in all instances other choices for the moiety; for example, a statement to the effect that R₁ “may be alkyl, aryl, or amino” does not necessarily exclude other choices for R₁, such as halo, aralkyl, and the like.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” refers to a value of 7.2 to 8.8, inclusive; as another example, the phrase “about 8%” refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” In another example, when a listing of possible substituents including “hydrogen, alkyl, and aryl” is provided, the recited listing may be construed as including situations whereby any of “hydrogen, alkyl, and aryl” is negatively excluded; thus, a recitation of “hydrogen, alkyl, and aryl” may be construed as “hydrogen and aryl, but not alkyl”, or simply “wherein the substituent is not alkyl”.

It has presently been discovered that certain adamantane variants are effective for inhibiting the respective viroporins of various virus species, including virus species in which a mutation of the viroporin and/or associated structures is present. Some of the adamantane variants concerning which this discovery has been made are commercially available, and others have been newly conceived by the present inventors. As used herein, “inhibition” of a viroporin refers to the reduction of the viroporin's ability to function in a manner that is most consistent with the vitality of the virus of which the viroporin is a component.

Accordingly, in one aspect, the present invention provides methods for treating an influenza virus-affected disease state or infection comprising the step of administering to a subject in need thereof a composition comprising a compound of Formula I:

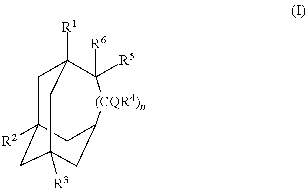

or a pharmaceutically acceptable salt thereof, wherein

R¹ is hydrogen, hydroxyl, halo, alkoxy, or together with R⁵ or R⁶ forms an optionally substituted five- or six-membered carbocyclic or heterocyclic ring;

R² and R³ are independently hydrogen, hydroxyl, halo, thiol, (C₁-C₃)alkylthio, or (C₁-C₃)alkoxy;

R⁴ is hydroxyl, oxo, oxime, amino, a three- to six-membered optionally substituted carbocyclic or heterocyclic ring, or together with R⁵ or R⁶ forms an aziridine group;

R⁵ and R⁶ are independently hydrogen, amino, nitro, cyano, hydroxyl, oxo, oximeamino(C₁-C₃)alkyl, hydroxy(C₁-C₃)alkylamino, formamidinyl, guanidinyl, oxime, —CH(X)(Y), a three- to six-membered heterocyclic ring, together with R⁴ forms an aziridine group, or together with R¹ forms an optionally substituted five- or six-membered carbocyclic or heterocyclic ring;

X and Y are independently hydrogen, hydroxyl, (C₁-C₃)alkyl, amino, amino(C₁-C₃)alkyl, (C₁-C₃)alkoxy, or (C₁-C₃)alkylamino;

Q is hydrogen or is absent; and,

n is 0 or 1;

wherein

if n=0, then at least one of R¹, R², R³, R⁵, and R⁶ is not hydrogen,
and,
if R³ is hydrogen, then at least one of the following conditions apply:
   n=1;
   either R⁵ or R⁶ comprises moiety that includes at least one carbon atom and at least one nitrogen atom and is other than an amino-substituted cyclohexane or dithiane; or,
   at least one of R⁵ and R⁶ is amino or nitro and R¹ is hydroxyl.

In certain embodiments, n=1. In some examples wherein n=1, R³ is hydrogen, halo, or hydroxyl. With respect to embodiments wherein n=1 and R³ is hydrogen, halo, or hydroxyl, it is sometimes the case that one of R⁵ and R⁶ is the same as R⁴. For example, one of R⁵ and R⁶ and R⁴ may both be oxime, amino, oxo, or hydroxyl.

With respect to further embodiments, R³ is hydroxyl or thiol. In some examples wherein R³ is hydroxyl or thiol, R¹ and R² may be hydrogen; R⁵ may be hydrogen; R⁶ may be amino, —CH(X)(Y), cyano, hydroxyl, or oxo; when n=1, R⁴ may be hydrogen, oxo, or hydroxyl; or, any combination of two or more of these conditions may apply. For example, in one embodiment, wherein R³ is hydroxyl or thiol, R¹ and R² are hydrogen; R⁵ is hydrogen; R⁶ is amino, —CH(X)(Y), cyano, hydroxyl, or oxo; n=1; and R⁴ is hydrogen, oxo, or hydroxyl.

In other embodiments, R³ is halo. Pursuant to some examples of this variety, R¹, R², R³, and R⁵ are hydrogen and n=0. When each of the preceding conditions apply, R⁶ may be, for example, formamidinyl, oxime, cyano, amino, or nitro.

Some embodiments are such that R³ is hydrogen. When this is the case, R¹ may also be hydrogen. In some examples when each of the preceding conditions apply, n=1 and R² is hydrogen or hydroxyl, and in some embodiments of this variety, R⁴ and R⁵ may each independently be amino, oxime, or oxo. In other examples wherein R³ is hydrogen, R¹ is also hydrogen, n=0, and R⁵ and R⁶ are independently hydrogen, hydroxyl, hydroxy(C₁-C₃)alkylamino, amino(C₁-C₃)alkyl, formamidinyl, or cyano.

R¹ may also be chosen such that that substituent together with R⁵ or R⁶ forms a five- or six-membered optionally substituted carbocyclic or heterocyclic ring. For example, R¹ may together with R⁵ or R⁶ form a five-membered heterocyclic ring that bears nitrogen, oxygen, or both nitrogen and oxygen heteroatoms.

Exemplary compounds according to formula I that may be used pursuant to the present methods include:

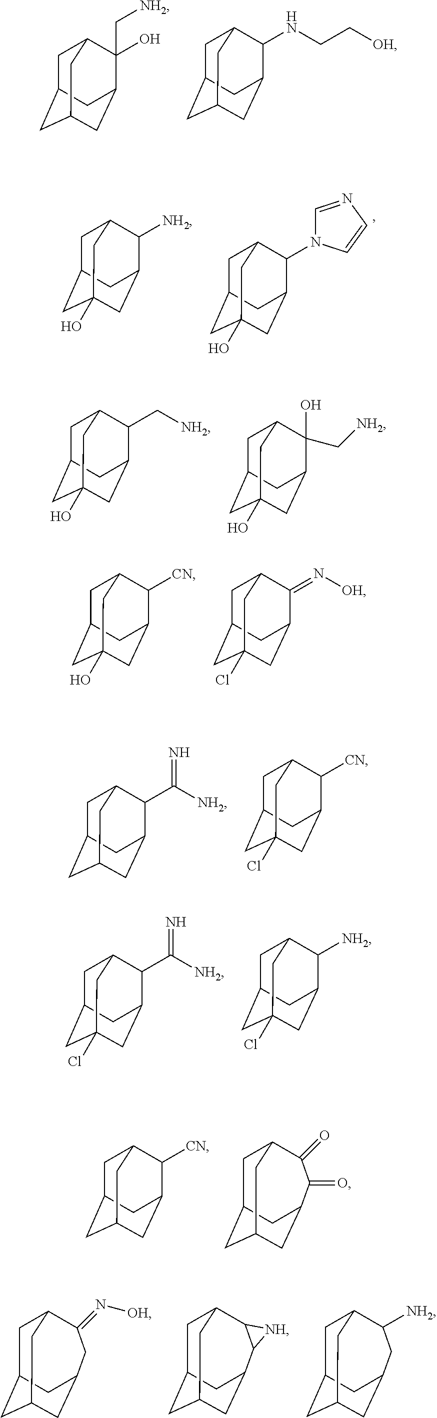

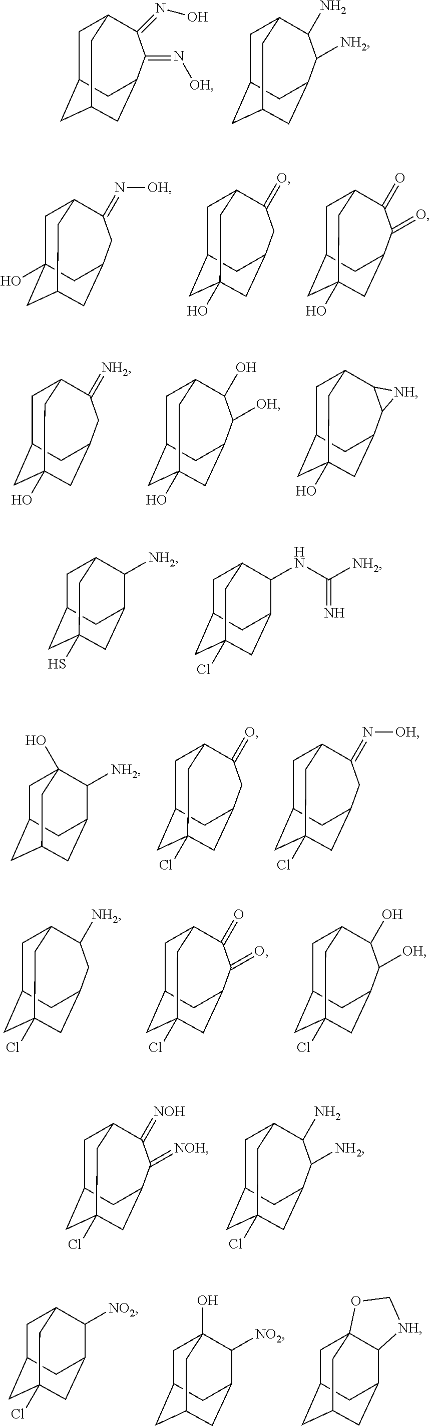

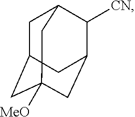

or a pharmaceutically acceptable salt or stereoisomer thereof.

In some embodiments, the methods provided herein inhibit an M2 proton channel (i.e., M2 protein or M2) of an influenza virus (including M2 of an influenza A virus and/or BM2 of an influenza B virus). In some embodiments, the M2 belongs to a wild type influenza virus. In some embodiments, the M2 belongs to an influenza virus strain that is resistant to the existing anti-influenza drugs (such as amantadine and/or rimantadine), for example, a S31N mutant. The mutant virus may comprise an influenza virus having the L26F mutation; may comprise an influenza virus having the V27G mutation, the V27I mutation, the V27T mutation, the V27S mutation, or the V27A mutation; may comprise an influenza virus having the A30T mutation; may comprise an influenza virus having the S31A mutation or the S31N mutation; may an influenza virus having the G34E mutation or the G34A mutation; may comprise an influenza virus having the L38F mutation; may comprise an influenza virus having the W41L mutation or the W41Y mutation; may comprise an influenza virus having the D44N mutation or the D44H mutation; and/or may comprise an influenza virus having the R45K mutation or the R45H mutation.

In some embodiments, the methods provided herein inhibit VP24 of an Ebola or a Marburg virus.

In some embodiments, the methods provided herein inhibit NS3 protein of a Bluetongue virus.

In some embodiments, the methods provided herein inhibit a viroporin of a picornavirus, foot and mouth disease virus, African horse sickness virus, or Japanese encephalitis virus.

In some embodiments, the compounds and/or salts provided herein can inhibit (i.e., decrease activity of) an M2 proton channel of an influenza virus (including M2 of an influenza A virus; BM2 of an influenza B virus, M2 of a wild type influenza virus, and/or M2 of a drug resistant influenza such as S31N influenza or other drug-resistant strains) by, for example, binding to the transmembrane region of M2 and interfering with proton conduction inside the virus and ultimately preventing the replication of the virus. In some embodiments, the compounds and/or salts provided herein can inhibit M2 and prevent viral maturation and release from the host cell. Accordingly, in some embodiments, the present invention provides a method for treating influenza (including wild type influenza and/or drug resistant influenza such as S31N influenza or other drug-resistant strains) in a patient (including a human or another animal) comprising contacting the patient with a therapeutically effective amount of a compound of Formula I as defined herein. In some embodiments, the method is a method for treating influenza that is a wild type. In some embodiments, the method is for treating influenza that is resistant to one or more of the existing anti-influenza drugs. In some embodiments, the method is a method for treating influenza that is resistant to amantadine and/or rimantadine.

In some embodiments, the compounds and/or salts provided herein can inhibit other integral membrane proteins that possess viroporin activity similar to the M2 protein (for example, VP24 of Ebola and Marburg viruses, NS3 protein of a Bluetongue virus, and a viroporin of a picornavirus, foot and mouth disease virus, African horse sickness virus, or Japanese encephalitis virus). Accordingly, in some embodiments, the present invention provides methods for treating Ebola, Marburg, bluetongue, foot and mouth disease, African horse sickness, and Japanese encephalitis in a patient (including a human or another animal) comprising contacting the patient with a therapeutically effective amount of the compound of Formula I as defined herein. In some embodiments, the method is a method for treating Ebola or Marburg in a patient. In some embodiments, the method is a method for treating Bluetongue in a patient. In some embodiments, the method is a method of treating a picornavirus infection, foot and mouth disease, African horse sickness, or Japanese encephalitis in a patient.

Methods of measuring inhibition of M2 protein of an influenza virus (or other integral membrane proteins that possess viroporin activity similar to the M2 protein (for example, VP24 of Ebola and Marburg viruses, NS3 protein of a Bluetongue virus, and a viroporin of a picornavirus, foot and mouth disease, African horse sickness, or Japanese encephalitis virus) are routine in the art.

The present invention further provides methods for treating viral infections such as influenza, Ebola, Marburg, bluetongue, foot and mouth disease, African horse sickness, and Japanese encephalitis in an individual (e.g., patient) by administering to the individual in need of such treatment a therapeutically effective amount or dose of a compound of Formula I as defined herein or a pharmaceutical composition thereof.

As used herein, the term “cell” is meant to refer to a cell that is in vitro, ex vivo or in vivo. In some embodiments, an ex vivo cell can be part of a tissue sample excised from an organism such as a mammal In some embodiments, an in vitro cell can be a cell in a cell culture. In some embodiments, an in vivo cell is a cell living in an organism such as a mammal In some embodiments, the cell is an adipocyte, a pancreatic cell, a hepatocyte, neuron, or cell of the eye.

As used herein, the term “contacting” refers to the bringing together of indicated moieties in an in vitro system or an in vivo system. For example, “contacting” the M2 protein (i.e., the M2 proton channel) of an influenza virus with a compound in the invention includes the administration of a compound in the present invention to an individual or patient, such as a human, having an influenza infection, as well as, for example, introducing a compound of the invention into a sample containing a cellular or purified preparation containing the M2 protein.

As used herein, the term “individual” or “patient,” used interchangeably, refers to any animal, including mammals, such as mice, rats, other rodents, rabbits, dogs, cats, swine, cattle, sheep, horses, or primates, such as humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease;

(2) inhibiting the disease; for example, inhibiting a disease, condition or disorder in an individual who is experiencing or displaying the pathology or symptomatology of the disease, condition or disorder (i.e., including arresting further development of the pathology and/or symptomatology); and

(3) ameliorating the disease; for example, ameliorating a disease, condition or disorder in an individual who is experiencing or displaying the pathology or symptomatology of the disease, condition or disorder (i.e., including reversing the pathology and/or symptomatology).

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, and the like, avian species, such as chickens, turkeys, songbirds, and the like, i.e., for veterinary medical use.

In other aspects, provided are compositions comprising a compound according to Formula I as defined herein or a pharmaceutically acceptable salt or stereoisomer thereof and a pharmaceutically acceptable carrier, diluent, or excipient. The applicable carrier, diluent, or excipient may be selected on the basis of the chosen route of administration and standard pharmaceutical practice as described, for example, in Remington's Pharmaceutical Sciences (Mack Pub. Co., Easton, Pa., 1985), the disclosure of which is hereby incorporated by reference in its entirety.

With respect to certain embodiments, the present compositions may further comprise a therapeutically effective amount of a further agent that modulates Influenza A virus, Influenza B virus, or another Viroporin-type virus. For example, the further agent that modulates virus may be a known anti-viral agents, such as Tamiflu®, Relenza®, or peramivir. In certain embodiments, the present compositions comprise a therapeutically effective amount of a compound of formula I which is administered in combination with immunizations or vaccines that are effective in preventing or lessening the symptoms of influenza. Examples include antibodies, immune suppressants, anti-inflammatory agents, and the like.

In another aspect of the present invention, provided are compounds of the formula II:

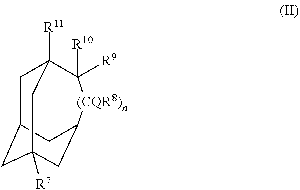

or a pharmaceutically acceptable salt thereof, wherein

n is 0 or 1;

R⁷ is hydroxyl or halo, or, if R¹¹ is hydroxyl or forms an optionally substituted five- or six-membered carbocyclic or heterocyclic ring together with R¹⁰, R⁷ may be hydrogen;

R⁸ is hydrogen, hydroxyl, amino, oxo, oxime, or together with R⁹ or R¹⁰ forms an aziridine group;

R⁹ is hydrogen, hydroxyl, cyano, amino, formamidinyl, guanidinyl, a three- to six-membered heterocyclic ring, or —CH(X)(Y);

if R⁷ is hydroxyl, then R¹⁰ is hydroxyl, cyano, formamidinyl, guanidinyl, a three- to six-membered heterocyclic ring, or —CH(X)(Y), or together with R⁸ forms an aziridine group, and if n is 1, R¹⁰ may additionally be amino;

if R⁷ is halo, then R¹⁰ is hydroxyl, nitro, formamidinyl, guanidinyl, a three- to six-membered heterocyclic ring, or —CH(X)(Y), and if n is 1, R¹⁰ may additionally be oxo, amino, oxime, or hydroxyl;

if R⁷ is hydrogen, then R¹⁰ is nitro, or together with R¹¹ forms an optionally substituted five- or six-membered carbocyclic or heterocyclic ring;

R¹¹ is hydrogen, hydroxyl, or together with R¹⁰ forms an optionally substituted five- or six-membered carbocyclic or heterocyclic ring;

X and Y are independently hydrogen, amino, amino(C₁-C₃)alkyl, or (C₁-C₃)alkylamino; and,

Q is hydrogen or is absent.

In some embodiments of the present compounds, n=0. In certain embodiments wherein n=0, R⁷ is hydroxyl. When both of such conditions apply, R⁹ may be, for example, hydrogen or hydroxyl. In such embodiments, R¹⁰ may be, for example, —CH(X)(Y), cyano, or formamidinyl.

In other embodiments of the present compounds, n=1. In certain embodiments wherein n=1, R⁷ is hydroxyl. When both of such conditions apply, R⁹ may be, for example, hydrogen, and R¹⁰ may be amino, hydroxyl, or, together with R⁸ forms an aziridine group.

Some compounds are such that R⁷ is halo. In such instances, R⁹ may be, for example, hydrogen. When R⁷ is halo and R⁹ is hydrogen, R¹⁰ may be, in some embodiments, oxo, amino, nitro, oxime, or hydroxyl. When all of these conditions apply, with respect to some of the present compounds, n=1 and R⁸ is hydrogen, oxo, hydroxyl, oxime, or amino.

There are also some embodiments of the present compounds wherein R⁷ is hydrogen. When this is the case, R¹¹ may be, for example, hydroxyl. When both of these conditions apply, it is possible for R¹⁰ to be nitro.

In certain instances R¹¹ together with R⁹ or R¹⁰ may form a five- or six-membered optionally substituted carbocyclic or heterocyclic ring. For example, R¹¹ may together with R⁹ or R¹⁰ form a five-membered heterocyclic ring that bears nitrogen, oxygen, or both nitrogen and oxygen heteroatoms.

Exemplary compounds according to formula I that may be used pursuant to the present methods include:

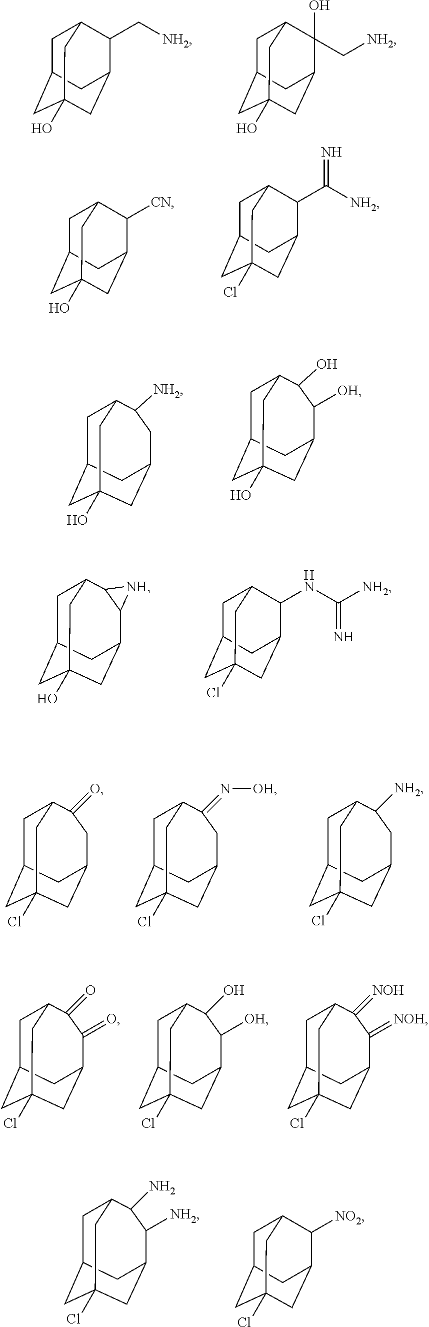

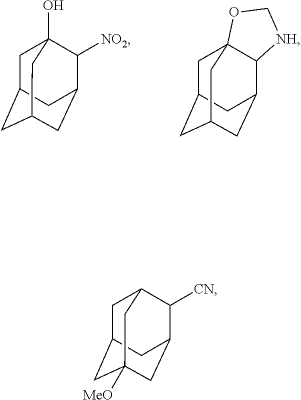

or a pharmaceutically acceptable salt or stereoisomer thereof.

The compounds of this invention may be administered orally or parenterally, neat or in combination with conventional pharmaceutical carriers, diluents, or excipients, which may be liquid or solid. The applicable solid carrier, diluent, or excipient may function as, among other things, a binder, disintegrant, filler, lubricant, glidant, compression aid, processing aid, color, sweetener, preservative, suspensing/dispersing agent, tablet-disintegrating agent, encapsulating material, film former or coating, flavors, or printing ink. Of course, any material used in preparing any dosage unit form is preferably pharmaceutically pure and substantially non-toxic in the amounts employed. In addition, the active compound may be incorporated into sustained-release preparations and formulations. Parenteral administration in this respect includes administration by, inter alia, the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol, and rectal systemic.

In powders, the carrier, diluent, or excipient may be a finely divided solid that is in admixture with the finely divided active ingredient. In tablets, the active ingredient is mixed with a carrier, diluent or excipient having the necessary compression properties in suitable proportions and compacted in the shape and size desired. For oral therapeutic administration, the active compound may be incorporated with the carrier, diluent, or excipient and used in the form of ingestible tablets, buccal tablets, troches, capsules, elixirs, suspensions, syrups, wafers, and the like. The amount of active compound(s) in such therapeutically useful compositions is preferably such that a suitable dosage will be obtained. The therapeutic compositions preferably contain up to about 99% of the active ingredient.

Liquid carriers, diluents, or excipients may be used in preparing solutions, suspensions, emulsions, syrups, elixirs, and the like. The active ingredient of this invention can be dissolved or suspended in a pharmaceutically acceptable liquid such as water, an organic solvent, a mixture of both, or pharmaceutically acceptable oils or fat. The liquid carrier, excipient, or diluent can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators.

Suitable solid carriers, diluents, and excipients may include, for example, calcium phosphate, silicon dioxide, magnesium stearate, talc, sugars, lactose, dextrin, starch, gelatin, cellulose, methyl cellulose, ethylcellulose, sodium carboxymethyl cellulose, microcrystalline cellulose, polyvinylpyrrolidine, low melting waxes, ion exchange resins, croscarmellose carbon, acacia, pregelatinized starch, crospovidone, HPMC, povidone, titanium dioxide, polycrystalline cellulose, aluminum methahydroxide, agar-agar, tragacanth, or mixtures thereof.

Suitable examples of liquid carriers, diluents and excipients for oral and parenteral administration include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil), or mixtures thereof.

For parenteral administration, the carrier, diluent, or excipient can also be an oily ester such as ethyl oleate and isopropyl myristate. Also contemplated are sterile liquid carriers, diluents, or excipients, which are used in sterile liquid form compositions for parenteral administration. Solutions of the active compounds as free bases or pharmacologically acceptable salts can be prepared in water suitably mixed with a surfactant, such as hydroxypropylcellulose. A dispersion can also be prepared in glycerol, liquid polyethylene glycols, and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier, diluent, or excipient may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compounds in the required amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and the freeze drying technique that yields a powder of the active ingredient or ingredients, plus any additional desired ingredient from the previously sterile-filtered solution thereof.

The compounds of the invention may be administered in an effective amount by any of the conventional techniques well-established in the medical field. The compounds employed in the methods of the present invention including, for example, the compounds of formula I may be administered by any means that results in the contact of the active agents with the agents' site or sites of action in the body of a patient. The compounds may be administered by any conventional means available.

Preferably the pharmaceutical composition is in unit dosage form, e.g. as tablets, buccal tablets, troches, capsules, elixirs, powders, solutions, suspensions, emulsions, syrups, wafers, granules, suppositories, or the like. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form. In addition, dosage forms of the present invention can be in the form of capsules wherein one active ingredient is compressed into a tablet or in the form of a plurality of microtablets, particles, granules or non-perils. These microtablets, particles, granules or non-perils are then placed into a capsule or compressed into a capsule, possibly along with a granulation of the another active ingredient.

The dosage of the compounds of the present invention that will be most suitable for prophylaxis or treatment will vary with the form of administration, the particular compound chosen and the physiological characteristics of the particular patient under treatment. Generally, small dosages may be used initially and, if necessary, increased by small increments until the desired effect under the circumstances is reached. Generally speaking, oral administration may require higher dosages.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The dose may also be provided by controlled release of the compound, by techniques well known to those in the art.

Additional information regarding the preparation of the present compounds for administration and the formulation of compostions according to the present invention is provided infra.

The compounds useful in the methods of the present invention may be prepared in a number of ways well known to those skilled in the art. The compounds can be synthesized, for example, by the methods as described below, or variations thereon as appreciated by the skilled artisan. The reagents used in the preparation of the compounds of this invention can be either commercially obtained or can be prepared by standard procedures described in the literature. All processes disclosed in association with the present invention are contemplated to be practiced on any scale, including milligram, gram, multigram, kilogram, multikilogram or commercial industrial scale.

For compounds herein in which a variable appears more than once, each variable can be a different moiety selected from the Markush group defining the variable. For example, where a structure is described having two R groups that are simultaneously present on the same compound, the two R groups can represent different moieties selected from the Markush group defined for R.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the terms “component,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

The abbreviations in the specification correspond to units of measure, techniques, properties, or compounds as follows: “min” means minute(s), “g” means gram(s), “mg” means milligram(s), “μg” means microgram(s), “eq” means equivalent(s), “h” means hour(s), “μL” means microliter(s), “mL” means milliliter(s), “mM” means millimolar, “M” means molar, “mmol” or “mmole” means millimole(s), “cm” means centimeters, “SEM” means standard error of the mean, and “IU” means International Units. “IC₅₀ value” or “IC₅₀” means dose of the compound which results in 50% alleviation or inhibition of the observed condition or effect.

As used herein, “alkyl” refers to an optionally substituted, saturated straight, or branched, hydrocarbon radical having from about 1 to about 20 carbon atoms (and all combinations and subcombinations of ranges and specific numbers of carbon atoms therein). Where appropriate, “alkyl” can mean “alkylene”; for example, if X is —R₁R₂, and R₁ is said to be “alkyl”, then “alkyl” may correctly be interpreted to mean “alkylene”. In the present disclosure, the term “alkyl” or a shortened or otherwise modified version thereof (for example, as used in conjunction with another substituent, for example, in the case of “alkoxy” or “aminoalkyl”) may be preceded by a range specifying the number of carbon atoms in the alkyl portion of the described moiety. For example, “amino(C₁-C₃)alkyl” refers to the fact that the alkyl portion of the substituent possesses one to three carbon atoms.

“Amino” refers to —NH₂ and may include one or more substituents that replace hydrogen. “Amino” is used interchangeably with amine and is also intended to include any pharmaceutically acceptable amine salts. For example, amino may refer to —NH⁺(X)(Y)Cl⁻, wherein X and Y are preferably and independently hydrogen or alkyl, wherein alkyl may include one or more halo substitutions.

As used herein, “aryl”, “arene”, and “aromatic” each refer to an optionally substituted, saturated or unsaturated, monocyclic, polycyclic, or other homo- or heterocyclic aromatic ring system having from about 3 to about 50 ring members (and all combinations and subcombinations of ranges and specific numbers of carbon atoms therein), with from about 5 to about 10 ring atom members being preferred. Such moieties encompass (include) “heteroaryl” and “heteroarene” as defined infra. Where appropriate, “aryl” can mean “arene”; for example, if X is —R₁R₂, and R₁ is said to be “aryl”, then “aryl” may correctly be interpreted to mean “arene”.

As used herein, “alkenyl” refers to an alkyl radical having from about 2 to about 20 carbon atoms and one or more double bonds (and all combinations and subcombinations of ranges and specific numbers of carbon atoms therein), wherein alkyl is as previously defined. In some embodiments, it is preferred that the alkenyl groups have from about 2 to about 6 carbon atoms. Alkenyl groups may be optionally substituted.

As used herein, “aralkyl” refers to alkyl radicals bearing one or more aryl substituents and having from about 4 to about 50 carbon atoms (and all combinations and subcombinations of ranges and specific numbers of carbon atoms therein), wherein aryl and alkyl are as previously defined. In some preferred embodiments, the alkyl moieties of the aralkyl groups have from about 1 to about 4 carbon atoms. In other preferred embodiments, the alkyl moieties have from about 1 to about 3 carbon atoms. Aralkyl groups may be optionally substituted.

“Alkylamino” signifies alkyl-(NH)—, wherein alkyl is as previously described and NH is defined in accordance with the provided definition of amino. “Arylamino” represents aryl-(NH)—, wherein aryl is as defined herein and NH is defined in accordance with the provided definition of amino. Likewise, “aralkylamino” is used to denote aralkyl-(NH)—, wherein aralkyl is as previously defined and NH is defined in accordance with the provided definition of amino “Alkylamido” refers to alkyl-CH(═O)NH—, wherein alkyl is as previously described. “Alkoxy” as used herein refers to the group R—O— where R is an alkyl group, and alkyl is as previously described. “Aralkoxy” stands for R—O—, wherein R is an aralkyl group as previously defined. “Alkylsulfonyl” means alkyl-SO₂—, wherein alkyl is as previously defined. “Aminooxy” as used herein refers to the group amino-(O)—, wherein amino is defined as above. “Aralkylaminooxy” as used herein is used to denote aryl-akyl-aminooxy-, wherein aryl, alkyl, and aminooxy are respectively defined as provided previously.

As used herein, “alkylene” refers to an optionally branched or substituted bivalent alkyl radical having the general formula —(CH₂)ₙ—, where n is 1 to 10. Non-limiting examples include methylene, trimethylene, pentamethylene, and hexamethylene.

“Alkyleneamino” refers to —(CH₂)ₙ—NH—, where n is 1 to 10 and wherein the bivalent alkyl radical may be optionally branched or substituted, and the amino group may include one or more substituents that replace hydrogen.

As used herein, “heteroaryl” or “heteroarene” refers to an aryl radical wherein in at least one of the rings, one or more of the carbon atom ring members is independently replaced by a heteroatom group selected from the group consisting of S, O, N, and NH, wherein aryl is as previously defined. Heteroaryl/heteroarene groups having a total of from about 3 to about 14 carbon atom ring members and heteroatom ring members are preferred. Likewise, a “heterocyclic ring” is an aryl radical wherein one or more of the carbon atom ring members may be (but are not necessarily) independently replaced by a heteroatom group selected from the group consisting of S, O, N, and NH. Heterocyclic rings having a total from about 3 to 14 ring members and heteroatom ring members are preferred, but not necessarily present; for example, “heterocyclohexyl” may be a six-membered aryl radical with or without a heteroatom group.

“Halo” and “halogen” each refers to a fluoro, chloro, bromo, or iodo moiety, with fluoro, chloro, or bromo being preferred.

“Haloalkyl” signifies halo-alkyl- wherein alkyl and halo, respectively, are as previously described.

“Thiol” refers to —SH. “Thionyl” refers to ═S.

“Nitroalkyl” refers to NO₂-alkyl- wherein alkyl is as previously described.

The phrase reading “[moiety] is absent” means that the substituents to which the moiety is attached may be directly attached to each other.

Typically, substituted chemical moieties include one or more substituents that replace hydrogen. Exemplary substituents include, for example, halo (e.g., F, Cl, Br, I), alkyl, cycloalkyl, alkylcycloalkyl, cycloalkylalkyl, alkenyl, alkynyl, aralkyl, aryl, heteroaryl, heteroaralkyl, spiroalkyl, heterocycloalkyl, hydroxyl (—OH), nitro (—NO₂), cyano (—CN), amino (—NH₂), —N-substituted amino (—NHR″), —N,N-disubstituted amino (—N(R″)R″), oxo (═O), carboxy (—COOH), —O—C(═O)R″, —C(═O)R″, —OR″, —C(═O)OR″, -(alkylene)-C(═O)—OR″, —NHC(═O)R″, aminocarbonyl (—C(═O)NH₂), —N-substituted aminocarbonyl (—C(═O)NHR″), —N,N-disubstituted aminocarbonyl (—C(═O)N(R″)R″), thiol (—SH), thiolato (—SR″), sulfonic acid (—SO₃H), phosphonic acid (—PO₃H), —P(═O)(OR″)OR″, —S(═O)R″, —S(═O)₂R″, —S(═O)₂NH₂, —S(═O)₂NHR″, —S(═O)₂NR″R″, —NHS(═O)₂R″, —NR″S(═O)₂R″, —CF₃, —CF₂CF₃, —NHC(═O)NHR″, —NHC(═O)NR″R″, —NR″C(═O)NHR″, —NR″C(═O)NR″R″, —NR″C(═O)R¹¹ and the like. In relation to the aforementioned substituents, each moiety R″ can be, independently, any of H, alkyl, cycloalkyl, alkenyl, aryl, aralkyl, heteroaryl, or heterocycloalkyl, for example.

As used herein, the terms “treatment” or “therapy” (as well as different word forms thereof) includes preventative (e.g., prophylactic), curative or palliative treatment.

As employed above and throughout the disclosure the term “effective amount” refers to an amount effective, at dosages, and for periods of time necessary, to achieve the desired result with respect to the treatment of the relevant disorder, condition, or side effect. It will be appreciated that the effective amount of components of the present invention will vary from patient to patient not only with the particular compound, component or composition selected, the route of administration, and the ability of the components to elicit a desired response in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage ultimately being at the discretion of the attendant physician. Dosage regimens may be adjusted to provide the improved therapeutic response. An effective amount is also one in which any toxic or detrimental effects of the components are outweighed by the therapeutically beneficial effects. As an example, the compounds useful in the methods of the present invention are administered at a dosage and for a time such that the level of activation and adhesion activity of platelets is reduced as compared to the level of activity before the start of treatment.

Compounds described herein throughout, can be used or prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein throughout that contain, for example, both amino and carboxy groups, also include reference to their corresponding zwitterions.

“Hydrate” refers to a compound of the present invention which is associated with water in the molecular form, i.e., in which the H—OH bond is not split, and may be represented, for example, by the formula R.H₂O, where R is a compound of the invention. A given compound may form more than one hydrate including, for example, monohydrates (R.H₂O) or polyhydrates (R.nH₂O wherein n is an integer >1) including, for example, dihydrates (R.2H₂O), trihydrates (R.3H₂O), and the like, or hemihydrates, such as, for example, R.n_{/2}H₂O, R.n_{/3}H₂O, R.n_{/4}H₂O and the like wherein n is an integer.

“Solvate” refers to a compound of the present invention which is associated with solvent in the molecular form, i.e., in which the solvent is coordinatively bound, and may be represented, for example, by the formula R.(solvent), where R is a compound of the invention. A given compound may form more than one solvate including, for example, monosolvates (R.(solvent)) or polysolvates (R.n(solvent)) wherein n is an integer >1) including, for example, disolvates (R.2(solvent)), trisolvates (R.3(solvent)), and the like, or hemisolvates, such as, for example, R.n_{/2}(solvent), R.n_{/3}(solvent), R.n_{/4}(solvent) and the like wherein n is an integer. Solvents herein include mixed solvents, for example, methanol/water, and as such, the solvates may incorporate one or more solvents within the solvate.

“Acid hydrate” refers to a complex that may be formed through association of a compound having one or more base moieties with at least one compound having one or more acid moieties or through association of a compound having one or more acid moieties with at least one compound having one or more base moieties, said complex being further associated with water molecules so as to form a hydrate, wherein said hydrate is as previously defined and R represents the complex herein described above.

As used herein, the terms “substitute” or “substitution” refer to replacing a hydrogen with a non-hydrogen moiety.

As used used herein, the term “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent group, provided that the normal valency of the designated atom or moiety is not exceeded, and that the substitution results in a stable compound. For example, if a methyl group (i.e., CH₃) is optionally substituted, then 3 hydrogens on the carbon atom can be replaced with substituent groups.

The compounds described herein can be asymmetric (e.g., having one or more stereocenters). All stereoisomers, such as enantiomers, epimers, and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention also include tautomeric forms, such as keto-enol tautomers.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds of the invention are intended to include compounds with stable structures. As used herein, “stable compound” and “stable structure” are meant to indicate a compound that is sufficiently robust to survive isolation to a useful degree of purity from a reaction mixture, and formulation into an efficacious therapeutic agent.

The phrase “pharmaceutically acceptable” is employed herein to refer to those compounds, materials, compositions, and/or dosage forms which are, within the scope of sound medical judgement, suitable for use in contact with the tissues of human beings and/or animals with acceptable levels of toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts or the quaternary ammonium salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in, for example, Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

The present invention also includes prodrugs of the compounds described herein. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation and use of prodrugs is discussed in, for example, T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

When any variable occurs more than one time in any constituent or in any formula, its definition in each occurrence may be independent of its definition at every other occurrence. Combinations of substituents and/or variables are permissible only if such combinations result in stable compounds.

The term “administering” means either directly administering a compound or composition of the present invention, or administering a prodrug, derivative or analog which will form an equivalent amount of the active compound or substance within the body.

“Dosage unit” refers to physically discrete units suited as unitary dosages for the particular individual to be treated. Each unit may contain a predetermined quantity of active compound(s) calculated to produce the desired therapeutic effect(s) in association with the required pharmaceutical carrier. The specification for the dosage unit forms of the invention may be dictated by (a) the unique characteristics of the active compound(s) and the particular therapeutic effect(s) to be achieved, and (b) the limitations inherent in the art of compounding such active compound(s).

“Subject” or “patient” refers to an embryonic, immature, or adult animal, including the human species, that is treatable with the compositions, and/or methods of the present invention.

The present invention is further defined in the following Examples. It should be understood that these examples, while indicating preferred embodiments of the invention, are given by way of illustration only, and should not be construed as limiting the appended claims From the above discussion and these examples, one skilled in the art can ascertain the essential characteristics of this invention, and without departing from the spirit and scope thereof, can make various changes and modifications of the invention to adapt it to various usages and conditions.

### General Synthesis

All chemicals for use in preparing the inventive compounds were purchased from commercial vendors and used without further purification, unless otherwise noted.

Synthesis of some preferred embodiments was accomplished as illustrated in the following generalized schematics and as described below:

The compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

The compounds of this invention can be prepared from readily available starting materials using the following general methods and procedures. It will be appreciated that where typical or suitable process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C NMR), infrared spectroscopy (IR), spectrophotometry (e.g., UV-visible), or mass spectrometry, or by chromatography such as high performance liquid chromatograpy (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of the invention can be prepared, for example, using the reaction pathways and techniques as described below.

As shown in Scheme 1, ketone 1-1 [PG is an —OH protecting group (such as -TES, -TBS)] can be reacted with TMSCN/ZnI₂ to form nitrile 1-2. Upon reduction by using a suitable reducing agent, such as metal aluminumhydride (e.g. lithium aluminumhydride), followed by the removal of protecting group (for example, under acidic conditions), alcohol 1-3 can be obtained in good yield. Reaction of nitrile 1-7 with AlMe₃/NH₄Cl gave amidine 1-8. Removal of protecting group of nitrile 1-4 provided alcohol 1-6.

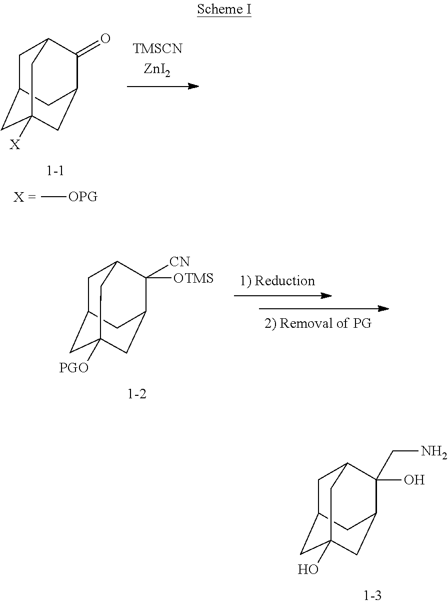

As shown in Scheme 2, nitrile 2-1, formed from the reaction of ketone 1-1 and TosMic/KOtBu, can be converted to amine 2-2 under the similar reduction/deprotection conditions as in Scheme 1. Removal of protecting group of nitrile 2-1 provided alcohol 2-3

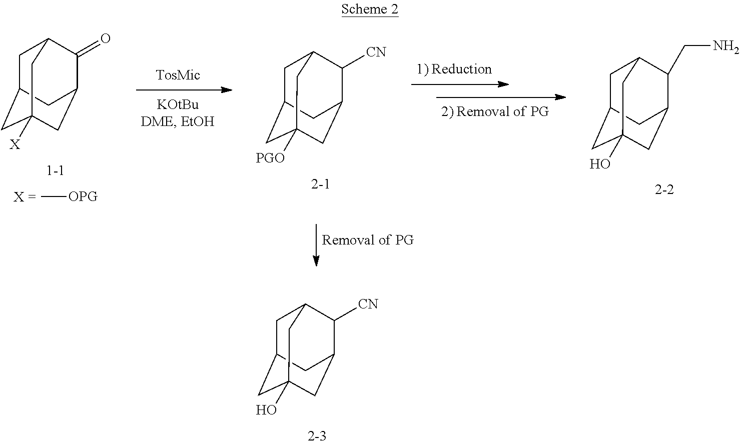

As shown in Scheme 3, reaction of nitrile 3-1, prepared from the reaction of ketone 1-1 and TosMic/KOtBu, with AlMe₃/NH₄Cl gave amidine 3-2.

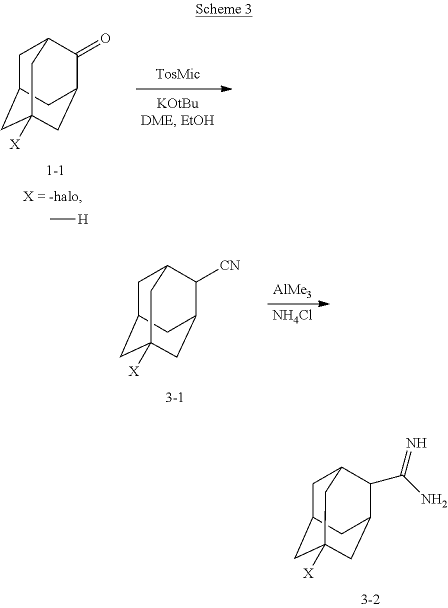

As shown in Scheme 4, conversion of ketone 4-1 to oxime 4-2 followed by a suitable reduction condition (e.g., in the presence of Raney Ni) can afford amine 4-3.

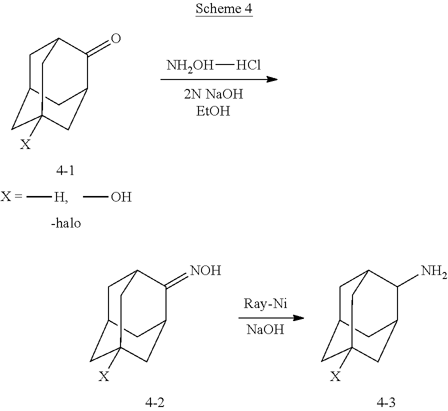

As shown in Scheme 5, ketone 4-1 can also be reacted with diazomethane (CH₂N₂) resulting in cycloalkanone ring expansion to afford ketone 5-1. Oxidation of ketone 5-1 provided diketone 5-2. Oxime 5-3 and amine 5-4 can be obtained under similar reaction sequence with compounds 4-2 and 4-3 as in Scheme 4.

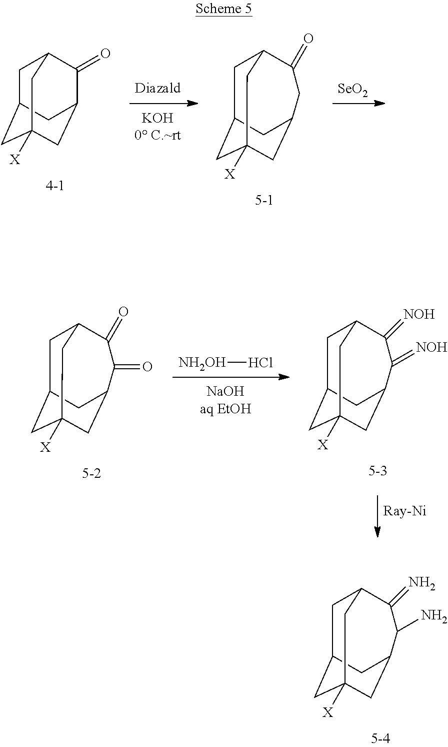

As shown in Scheme 6, reduction of diketone 5-2 by using a suitable reducing agent, such as metal borohydride (e.g. sodium borohydride), resulted in the formation of diol 6-1.

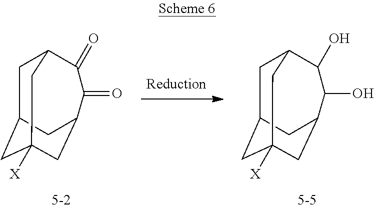

Additional starting materials and intermediates useful for making the compounds of the present invention can be obtained from chemical vendors such as Sigma-Aldrich or can be made according to methods described in the chemical art.

Those skilled in the art can recognize that in all of the schemes described herein, functional (reactive) groups (including those on a substituent group such as X², X³ , Y¹, Y², etc., if present) can undergo further modification if appropriate and/or desired. For example, an OH group (such as the one in compound 1-1 or 2-3) can be converted into a better leaving group such as mesylate, which in turn is suitable for nucleophilic substitution, such as by CN or an azide group. For another example, a CN group can be hydrolyzed to afford an amide group or a carboxylic acid group; a carboxylic acid can be converted to an amide (for example, by standard coupling reactions with another amine (such as in the presence of an amide coupling reagent such BOP, HBTU, HATU, EDC, or DCC), and in the presence of a suitable base such as triethylamine, diisopropylethylamine, N-methylmorpholine, or N—N-dimethylaminopyridine); a carboxylic acid can be converted to an ester, which in turn can be reduced to an alcohol, which in turn can be further modified. For another example, an azide group can be reduced to an amino group. In some embodiments, a primary amine or a secondary amine moiety (present on a part of the compound of invention) can be converted to amide, sulfonamide, urea, or thiourea moiety by reacting it with an appropriate reagent such as an acid chloride, a sulfonyl chloride, an isocyanate, or a thioisocyanate compound. In some embodiments, a primary amine, a secondary amine, or a tertiary amine moiety (such as those present on part of the compound of invention) can be alkylated to form a quaternary ammonium salt. One skilled in the art will recognize further such modifications.

The compounds provided herein that contain one or more chiral centers can be prepared as racemates or mixtures of various stereoisomers. The stereoismers can further be separated. In addition, individual stereisomer can be prepared by chiral synthesis known to those skilled in the art. Different steroisomers may differ in pharmacological activity.

As used herein, the term “reacting” refers to the bringing together of designated chemical reactants such that a chemical transformation takes place generating a compound different from any initially introduced into the system. Reacting can take place in the presence or absence of solvent.

As provided above, qhen employed as pharmaceuticals, the compounds of the invention can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

The present invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 1 to about 100 mg, from about 5 to about 75 mg, from about 5 to about 50 mg, from about 10 to about 30 mg, or from about 10 to about 20 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral adminstration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents (including Tamiflu), antibodies, immune suppressants, anti-inflammatory agents, and the like.

### Labeled Compounds and Assay Methods

Another aspect of the present invention relates to labeled compounds of the invention (radio-labeled, fluorescent-labeled, colloidal gold-labeled, etc.) that would be useful not only in radio-imaging but also in assays, both in vitro and in vivo, for localizing and quantitating the virus in tissue samples, including human, and for identifying binding sites by inhibition binding of a labeled compound. Accordingly, the present invention includes enzyme assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from ³H, ¹⁴C, ¹²⁵I, ³⁵S and ⁸²Br.

In some embodiments, the labeled compounds of the present invention contain a fluorescent label, which are widely known to those skilled in the art.

Synthetic methods for incorporating radio-isotopes and fluorescent labels into organic compounds are are well known in the art.

A labeled compound of the invention (radio-labeled, fluorescent-labeled, etc.) can be used in a screening assay to identify/evaluate compounds. For example, a newly synthesized or identified compound (i.e., test compound) which is labeled can be evaluated for its ability to bind a virus by monitering its concentration variation when contacting with the virus, through tracking the labeling. For another example, a test compound (labeled) can be evaluated for its ability to reduce binding of another compound which is known to bind to a virus (i.e., standard compound). Accordingly, the ability of a test compound to compete with the standard compound for binding to the virus (e.g. M2 protein of an influenza virus) directly correlates to its binding affinity. Conversely, in some other screening assays, the standard compound is labled and test compounds are unlabeled. Accordingly, the concentration of the labled standard compound is monitored in order to evaluate the competition between the standard compound and the test compound, and the relative binding affinity of the test compound is thus ascertained.

### Kits

The present invention also includes pharmaceutical kits useful, for example, in the treatment or prevention of a viral infection disorder referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results. Certain compounds of the Examples were found to be inhibitors of viroporins (e.g. M2 protein of an influenza virus) according to one or more of the assays provided herein.

### EXAMPLES

All NMR spectra were recorded at 300 MHz on a Bruker Instruments NMR unless otherwise stated. Coupling constants (J) are in Hertz (Hz) and peaks are listed relative to TMS (δ 0.00 ppm). Microwave reactions were performed using a CEM Discovery™ microwave reactor in 2.5 mL or 5 mL microwave reactor vials. All reactions were performed at 200° C. for 600 s with the fixed hold time ON unless otherwise stated. TLC analysis was performed using Aldrich 254 nm plates (60 Å, 250 υm) and visualized using UV, PMA and KMnO4 stains. Unless otherwise disclosed, the reagents and solvents used in the preparation of the following examples were purchased from commercial sources (Aldrich, VWR, etc.) and used as received. Analytical HPLC/MS was performed on a 3 mm×50 mm Pursuit 3 Diphenyl column using a gradient of, typically, 5/95 to 100/0 acetonitrile (0.1% formic acid)/water (0.1% formic acid) over 7 min.

### Example 1

### Commercially Available through ChemBridge Corp. CAS 28529-71-7

### Example 2

### Commercially Available through ChemBridge Corp. CAS 24161-70-4

### Example 3

### Commercially Available through ChemBridge Corp. CAS 20098-19-5

### Example 4

### 4-imidazol-1-yladamantan-1-ol (4)

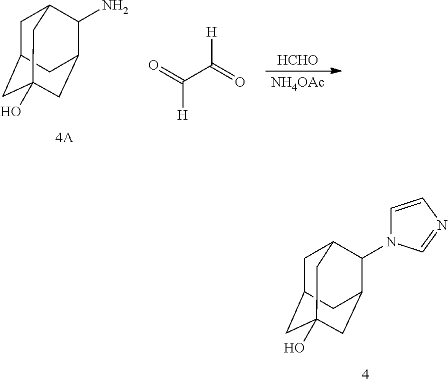

4-aminoadamantan-1-ol (342.0 mg, 4A) in 1.5 ml of dioxane was added to a mixture of 0.42 ml of glyoxal, 60.2 mg of paraformaldehyde, 515.6 mg of NH₄OAc and 1.5 ml of HOAc. The resulting mixture was irradiated under microwave at 165° C. for 20 minutes. Then the reaction mixture was cooled to 0° C. and quenched with 2N NaOH aqueous solution. The solution was then extracted with EtOAc twice and the organic phases were combined, dried (over anhydrous Na₂SO₄), and concentrated. The crude mixture was purified by column chromatography (0˜12% MeOH in EtOAc) to yield 132.6 mg of the desired product 4. ¹H NMR (d, ppm., DMSO): 7.75 (s, 1H, 1H), 7.24 (s, 1H, 1H), 6.90 (s, 1H, 1H), 4.49˜4.60 (m, 1H, 1H), 3.98˜4.10 (m, 1H, 1H), 2.55˜2.70 (m, 1H, 1H)), 1.45˜2.05 (m, 12H, 12H). ¹H NMR spectroscopy shows a 1:1 mixture of two isomers. LC-MS (ESR): m/z=219.3 (M+H)⁺.

### Example 5

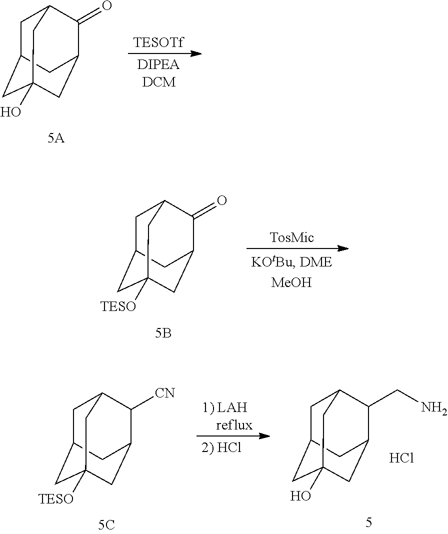

### Step 1. Synthesis of 5B

To a mixture of ketone 5B (1.5678 g), DIPEA (2.60 ml) in 20 ml of DCM, with stirring, was added a solution of TESOTf (1.65 ml) in DCM (5.0 ml) dropwise at −78° C. over a period of 15 min. Stirring was continued for 1 h at −78° C. and then for 2 h at 0° C. The reaction solution was slowly brought to room temperature (rt) and stirred for overnight. The reaction mixture was quenched with water, and then extracted with EtOAc twice and the organic phases were combined, dried (over anhydrous Na₂SO₄), and concentrated. The crude mixture was purified by column chromatography (0˜10% EtOAc in hexane) to yield 2.3405 g of the desired product 5B. LC-MS (ESR): m/z=281.1 (M+H)⁺.

### Step 2. Synthesis of 5C.

To a mixture of ketone 5B (1.1856 g), TosMic (1.4009 g) and 0.75 ml of anhydrous MeOH in 18 ml of DME, with stirring, was added KOtBu (1.1060 g) in five portions at 0° C. over a period of 30 min. Stirring was continued for 30 min at room temperature and then for an additional 30 min at 35 to 40° C. The reaction mixture was filtered. The solid was washed with EtOAc. The combined organic phase was washed with water, and saturated NaCl solution. After dried (over anhydrous Na₂SO₄), and concentrated, the crude mixture was purified by column chromatography (0˜40% EtOAc in hexane) to yield 0.9625 g of the desired product 5C as a mixture of diastereomers. LC-MS (ESR): m/z=292.3 (M+H)⁺.

### Step 2. Synthesis of 5.

To a 2.0 ml of 2.0 M LAH in THF was added 162.7 mg of the diastereomer mixture nitriles 5C and the reaction mixture was heated to reflux for overnight. The excess LAH was destructed by dropwise addition of 0.15 ml of water followed by dropwise addition of 0.15 ml of 15% of NaOH and subsequent addition of 0.45 ml of water. Stirring was continued for 3 h. Filtration yielded a clear solution, which was extracted with EtOAc twice and the organic phases were combined, dried (over anhydrous Na₂SO₄), and concentrated. The HCl salts of amines 5 were formed as a diastereomer mixture by the addition of HCl in ether to precipitate the 136.2 mg of the HCl salts. LC-MS (ESR): m/z=182.2 (M+H)⁺.

### EXAMPLES 6 AND 7

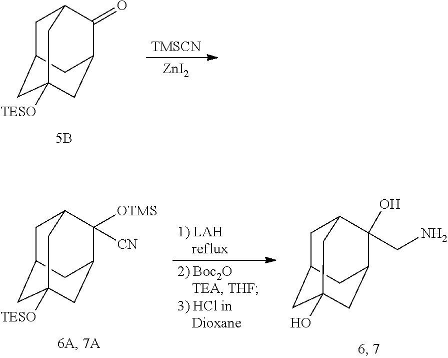

### Step 1. Synthesis of 6A, 7A

To a mixture of ketone 5B (252.8 mg) and ZnI₂ (3.5 mg) in 5 ml of DCM was added 0.20 ml of TMSCN dropwise at 0° C. over a period of 10 min. The reaction solution was slowly brought to room temperature (rt) and continued stirring for 3 days. The reaction mixture was concentrated and the crude products 6A, 7A were obtained as a diastereomer mixture and carried over to the next step without further purification.

### Step 2. Synthesis of 6 and 7

To a 199.2 mg of the crude diastereomer mixture of 6A, 7A in 10 ml of THF obtained from the previous step was added 0.8 ml of 2.0 M of LAH in THF dropwise and the resulting mixture was heated to reflux for overnight. The reaction was quenched by dropwise addition of 0.15 ml of water followed by dropwise addition of 0.15 ml of 15% of NaOH and subsequent addition of 0.45 ml of water. Stirring was continued for 3 h. Filtration yielded a clear solution, which was extracted with EtOAc twice and the organic phases were combined, dried (over anhydrous Na₂SO₄), and concentrated.

The crude oil was dissolved in 5 ml of THF. Boc-anhydride (196.6 mg), and triethylamine (TEA, 0.5 ml) was added sequentially and the resulting mixture was stirred at rt for 2 hrs before it was quenched with NaHCO₃ aqueous solution. The aqueous mixture was extracted with EtOAc twice. The combined organic phase was dried (anhydrous Na₂SO₄), and concentrated. The crude mixture was purified by column chromatography (0˜25% EtOAc in hexane) to yield 41.3 mg and 70.6 mg of the Boc- and TES-protected 6 (less polar isomer) and 7 (more polar isomer), respectively. LC-MS (ESR): Boc- and TES-protected 6 (less polar isomer): m/z=412.2 (M+H)⁺, 434.5 (M+Na)⁺; Boc- and TES-protected 7 (more polar isomer): m/z=412.3 (M+H)⁺, 434.3 (M+Na)⁺.

A solution of HCl/dioxane (2 ml, 4.0 M) was added to the 30.5 mg and 62.0 mg of Boc- and TES-protected 6 (less polar isomer) and 7 (more polar isomer), respectively, in one portion with stirring. After 4 h, LC-MS indicated that the reactions were completed. Each of the reaction mixture was concentrated by rotary evaporation under high vacuum at rt and then washed with dry ether three times. The white solid 6 and 7 formed were under high vacuum for overnight with the yields of 19.5 mg and 42.3 mg, respectively. LC-MS (ESR): 6: m/z=198.3 (M+H)⁺; 7: m/z=198.2 (M+H)⁺.

### EXAMPLES 8 AND 9

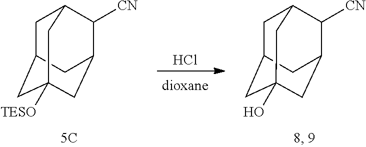

To a 2.0 ml of 4.0 M HCl in dioxane was added 71.6 mg of the diastereomer mixture nitriles 5C and the reaction mixture was stirred at rt for overnight. The reaction mixture was concentrated by rotary evaporation under high vacuum and the residue was purified by column chromatography (0˜75% EtOAc in DCM) to yield 20.6 mg and 19.8 mg of 8 (less polar isomer) and 9 (more polar isomer), respectively. LC-MS (ESR): 8: m/z=200.1 (M+Na)⁺; 9: m/z=200.1 (M+Na)⁺.

### Example 10

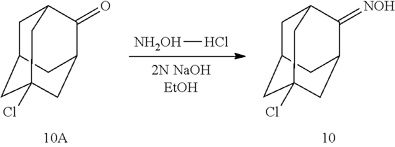

A solution of chloroketone 10A (736.6 mg) in 11 ml of EtOH (95%) was added to a mixture of 1.6443 g of NH₂OH—HCl in 6.5 ml of 2N NaOH. The resulting mixture was irradiated under microwave at 120° C. for 20 min. The reaction mixture was concentrated by rotary evaporation under high vacuum and the residue was extracted with EtOAc twice. The organic phases were combined, dried (over anhydrous Na₂SO₄), and concentrated. The residue was purified by column chromatography (0˜45% EtOAc in hexane) to yield 720.6 mg oxime 10 as an off white solid. LC-MS (ESR): m/z=200.1 (M+H)⁺.

### EXAMPLE 11

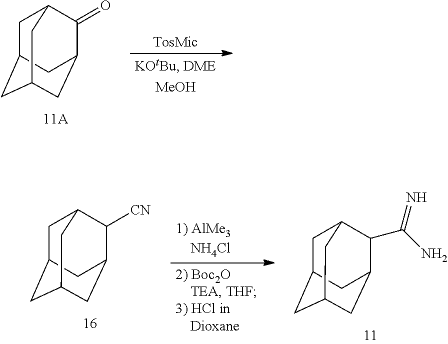

### Step 1. Synthesis of 16.

The compound 16 was prepared similarly as the transformation of 5B to 5C in Example 5.

### Step 2. Synthesis of 11.

A 2.0M solution of AlMe₃ (2.5 ml, 5 mmol) in toluene was slowly added to a suspension of 302.2 mg of NH₄Cl in 2.0 ml of toluene at 0° C. After the addition, the mixture was warmed to rt and stirred for 2 h. Then, 480.0 mg of nitrile 16 in 1.5 ml of toluene was added and the solution was heated to 80° C. for 20 h. The reaction mixture was slowly poured to a slurry of 1.5 g of silica gel in 5 ml of CHCl₃ and stirred for 15 min. The silica gel was filtered and washed with MeOH. The filtrate and wash were combined and concentrated to yield the crude amidine 11 that contains NH₄Cl salt.

The crude amidine 11 was suspended in 20 ml of THF. Boc-anhydride (596.6 mg), and triethylamine (TEA, 3.0 ml) was added sequentially and the resulting mixture was stirred at rt for 2 hrs before it was quenched with NaHCO₃ aqueous solution. The aqueous mixture was extracted with EtOAc twice. The combined organic phase was dried (anhydrous Na₂SO₄), and concentrated. The crude mixture was purified by column chromatography (0˜20% EtOAc in hexane) to yield 626.0 mg of Boc-protected 11.

A solution of HCl/dioxane (3 ml, 4.0 M) was added to the Boc-protected 11 (115.0 mg) in one portion with stirring. After overnight stirring, LC-MS indicated that the reaction was completed. The reaction mixture was concentrated by rotary evaporation under high vacuum at rt. The residue was then washed with dry ether three times. The white solid yielded (11) was under high vacuum for overnight. Yield is 89.1 mg. LC-MS (ESR): m/z=179.2 (M+H)⁺.

### EXAMPLE 12 AND 13

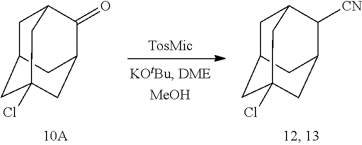

The compounds 12 and 13 were prepared similarly as the transformation of 5B to 5C in Example 5. Nitriles 12 (less polar isomer) and 13 (more polar isomer) were carefully separated over the column chromatography (0˜40% EtOAc in hexane). LC-MS (ESR): 8: m/z=218.1 (M+Na)⁺; 9: m/z=218.1 (M+Na)⁺.

### EXAMPLES 14 AND 17

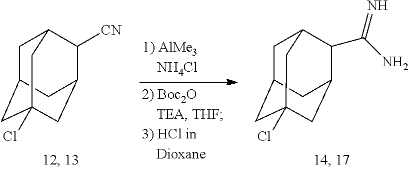

The compounds 14 and 17 were prepared similarly as the transformation of 16 to 11 in Example 11.

### EXAMPLE 15

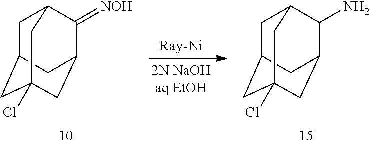

To a mixture of 327.6 mg of oxime 10 in 4.5 ml of 2N NaOH and 4.5 ml of EtOH, with stirring, was added 396.1 mg of Raney-Ni in portions at 0° C. over a period of 15 minutes. Stirring was continued overnight at rt and then was filtered off through celite. The filtrate was evaporated to dryness in vacuo and extracted with EtOAc twice. The combined organic phase was dried over Na₂SO₄. The solvent was evaporated to yield 236.1 mg of amine 15. LC-MS (ESR): 15: m/z=186.1(M+H)⁺.

### EXAMPLE 16

The compound 16 was prepared similarly as the transformation of 5B to 5C in Example 5.

### EXAMPLE 18

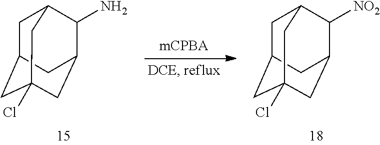

To an 86.2 mg of amine 15 in 6 ml of DCE was added 449.0 mg of mCPBA (up to 78% pure) and the reaction mixture was heated to reflux for 6 h. The reaction mixture was diluted with DCM and washed sequentially with saturated NaHCO₃ solution, NaHSO₃ solution, and saturated NaHCO₃ solution. The organic phase was dried (over anhydrous Na₂SO₄), and concentrated. The crude mixture was purified by column chromatography (0˜50% EtOAc in hexane) to yield 45.2 mg of desired product 18 as a mixture of diastereomers. ¹H NMR (300 MHz, CDCl₃) δ 4.05 (m, 1H), 2.60-1.19 (m, 13H).

### EXAMPLE 19

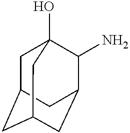

The compound 19 was prepared according to the reference of Rohde, J. J. et al. J. Med. Chem., 2007, 50 149-164.

### EXAMPLE 20

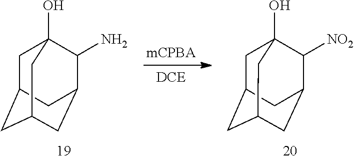

A mixture of 19 (334 mg, 2 mmol) and mCPBA (1.3 g, 70% pure, 7.5 mmol) in DCE (20 mL) was heated at 80° C. for 2 h and cooled to rt. The mixture was diluted with DCM (10 mL) and washed with saturated Na₂S₂O₃ and Na₂CO₃ (3×10 ml). The organic layer was dried over Na₂SO₄. Solvent was removed under vacuum to give a crude nitro 20 (276 mg) without further purification. ¹H NMR (300 MHz, CDCl₃) δ 3.85 (m, 1H), 2.93-1.64 (m, 13).

### EXAMPLE 21

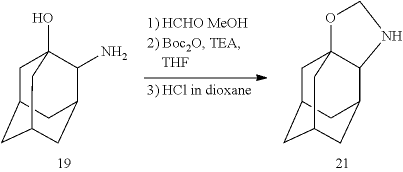

To a 68.6 mg of amine 19 in 2.5 ml of MeOH was added 67.1 mg of paraformaldehyde and the reaction mixture was stirred at rt for 24 h. The reaction mixture was concentrated. The crude amine 21 was suspended in 10 ml of THF. Boc-anhydride (96.6 mg), and triethylamine (TEA, 0.5 ml) was added sequentially and the resulting mixture was stirred at rt for 2 hrs before it was quenched with NaHCO₃ aqueous solution. The aqueous mixture was extracted with EtOAc twice. The combined organic phase was dried (anhydrous Na₂SO₄), and concentrated. The crude mixture was purified by column chromatography (0˜20% EtOAc in hexane) to yield 13.5 mg of Boc-protected 19.

A solution of HCl/dioxane (2 ml, 4.0 M) was added to the Boc-protected 19 (11.5 mg) in one portion with stirring. After overnight stirring, LC-MS indicated that the reaction was completed. The reaction mixture was concentrated by rotary evaporation under high vacuum at rt. The residue was then washed with dry ether three times. The white solid yielded (19) was under high vacuum for overnight. Yield is 6.2 mg. LC-MS (ESR): m/z=180.1 (M+H)⁺.

### EXAMPLE 22

### Commercially Available through ChemBridge Corp. CAS 1185341-09-6

### EXAMPLES 23 AND 24

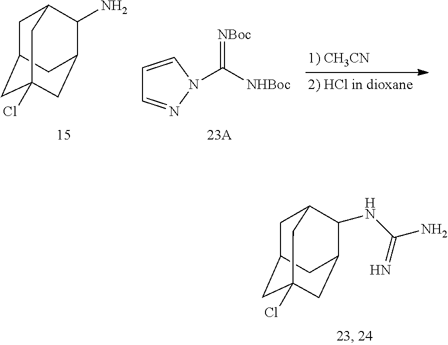

To a 99.2 mg of the diastereomer mixture of amines 15 in 12 ml of CH₃CN was added 274.6 mg of 23A in one portion and the resulting mixture was stirred at rt for overnight. The reaction was quenched with water followed by extraction of EtOAc twice and the organic phases were combined, dried (over anhydrous Na₂SO₄), and concentrated. The crude residue was purified by column chromatography (0˜10% EtOAc in hexane) to yield 22.3 mg and 28.6 mg of the Boc-protected 23 (less polar isomer) and 24 (more polar isomer), respectively. LC-MS (ESR): Boc-protected 23 (less polar isomer): m/z=428.3 (M+H)⁺, 450.2 (M+Na)⁺; Boc-protected 24 (more polar isomer): m/z=428.2 (M+H)⁺, 450.4 (M+Na)⁺.

A solution of HCl/dioxane (2 ml, 4.0 M) was added to the Boc-protected 23 (20.1 mg) in one portion with stirring. After 4 h, LC-MS indicated that the reaction was completed. The reaction mixture was concentrated by rotary evaporation under high vacuum at rt. The residue was then washed with dry ether three times. The white solid yielded (23) was under high vacuum for overnight. Yield is 13.3 mg. LC-MS (ESR): m/z=228.1 (M+H)⁺.

Compound 24 was prepared under the similar conditions. LC-MS (ESR): m/z=228.3 (M+H)⁺.

### EXAMPLE 25

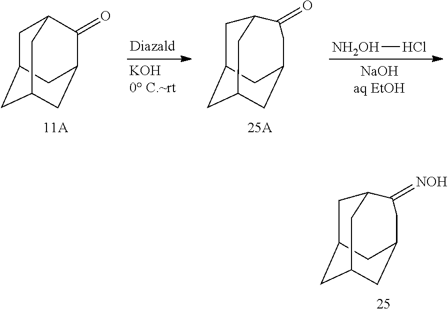

### Step 1. Synthesis of 25A.

To a mixture of ketone 11A (1.3658 g), KOH (5.4944 g), MeOH (20 ml) and water (3.5 ml), with stirring, was added a solution of diazomethane (Diazald, 4.75 g) in MeOH (35 ml) dropwise at 0° C. over a period of 1.5 hours. Stirring was continued overnight at room temperature (rt). The resulting suspension was evaporated to dryness in vacuo, and water (50 ml) was added. The resulting aqueous suspension was extracted with ether and DCM twice. Combined organic phase were dried over Na₂SO₄. The solvent was evaporated to yield 1.2050 g of 25A as a white solid. LC-MS (ESR): m/z=165.1 (M+H)⁺.

### Step 2. Synthesis of 25.

A solution of ketone 25A (336.4 mg) in 2.0 ml of EtOH (95%) was added to a mixture of 827.8 mg of NH₂OH—HCl in water (2.5 ml) and 233.6 mg of NaOH in 1.5 ml of water. The resulting mixture was irradiated under microwave at 120° C. for 20 min. The reaction mixture was then cooled to 0° C. On cooling, the oxime 25 crystallized and was filtered off, to yield 297.2 mg of oxime 25 as an off white solid. LC-MS (ESR): m/z=180.0 (M+H)⁺.

### EXAMPLE 26

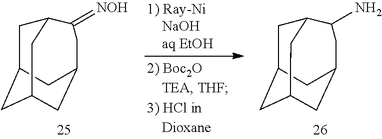

To a mixture of a solution of oxime 25 (72.0 mg), 2N NaOH (1.0 ml) and EtOH (1.0 ml), with stirring, was added Raney-Ni (87.0 mg) in portions at 0° C. over a period of 15 minutes. Stirring was continued overnight at rt and then was filtered off through celite. The filtrate was evaporated to dryness in vacuo and extracted with DCM twice. The combined organic phase was dried over Na₂SO₄. The solvent was evaporated to yield a crude material (oil).

The crude oil was dissolved in 5 ml of THF. Boc-anhydride (106.5 mg), and triethylamine (TEA, 0.5 ml) was added sequentially and the resulting mixture was stirred at rt for 2 hrs before it was quenched with NaHCO₃ aqueous solution. The aqueous mixture was extracted with EtOAc twice. The combined organic phase was dried (anhydrous Na₂SO₄), and concentrated. The crude mixture was purified by column chromatography (0˜5% EtOAc in hexane) to yield 78.7 mg of the Boc-protected 26. LC-MS (ESR): m/z=266.2 (M+H)⁺, 288.2 (M+Na)⁺.

2.0 ml of TFA was added to the Boc-protected 26 (37.1 mg) in 2.0 ml of DCM in one portion with stirring at rt. After 120 minutes, LC-MS indicated that the reaction was completed. The reaction mixture was concentrated by rotary evaporation under high vacuum at rt. The residue was then washed with dry ether three times. The white solid yielded (26) was under high vacuum for overnight. Yield is 26.7 mg. LC-MS (ESR): m/z=166.1 (M+H)⁺.

### EXAMPLE 27

The compound 27 was prepared according to the reference of Schlatmann, J. L. M. A. et al. Tetrahedron. 1970, 26, 949. LC-MS (ESR): m/z=164.1 (M+H)⁺.

### EXAMPLE 28

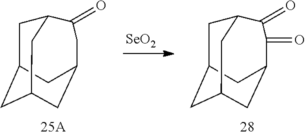

A mixture of ketone 25A (305.2 mg), SeO₂ (272.4 mg) in 3.0 ml of dioxane and 0.3 ml of water was stirred at 105° C. for overnight, then cooled to rt, filtered and the filtrate was evaporated in vacuo to dryness to give crude diketone 28. The crude mixture was purified by column chromatography (0˜75% EtOAc in hexane) to yield 302.1 mg of diketone 28. LC-MS (ESR): m/z=179.1 (M+H)⁺.

### EXAMPLE 29

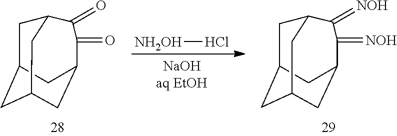

The compound 29 was prepared similarly as the transformation of 25A to 25 in Example 25. LC-MS (ESR): m/z=209.2 (M+H)⁺, 231.1 (M+Na)⁺.

### EXAMPLE 30

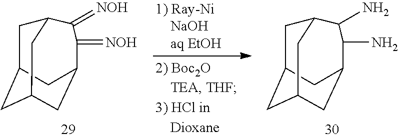

The compound 30 was prepared similarly as the transformation of 25 to 26 in Example 26. LC-MS (ESR): m/z=181.3 (M+H)⁺.

### EXAMPLES 31, 32, 33, 34 AND 35

The compounds 31, 32, 33, 34 and 35 were prepared similarly as the examples 25A, 25, 26, 27 and 28, respectively. LC-MS (ESR): 31: m/z=181.1 (M+H)⁺; 32: m/z=195.9 (M+H)⁺. 33: m/z=182.3 (M+H)⁺; 34: m/z=180.1 (M+H)⁺; 35: m/z=195.2 (M+H)⁻.

### EXAMPLE 36

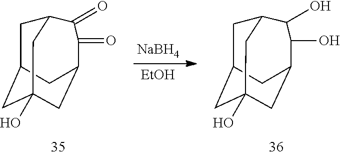

To a 36.1 mg of the diketone 35 in 3.0 ml of absolute ethanol was added 38.5 mg of NaBH₄ in two portions and the resulting mixture was stirred for 1 h at rt. The reaction was quenched with water followed by extraction of EtOAc twice and the organic phases were combined, dried (over anhydrous Na₂SO₄), and concentrated. The crude residue was purified by column chromatography (0˜15% MeOH in EtOAc) to yield 9.5 mg triol 23 as a mixture of diastereomers. LC-MS (ESR): 36: m/z=199.1 (M+H)⁺.

### EXAMPLES 37, 38, 39, 40, 41, 42, AND 43

The compounds 37, 38, 39, 40, 41, 42, and 43 were prepared similarly as the examples 25A, 25, 26, 28, 36, 29, and 30, respectively. LC-MS (ESR): 37: m/z=199.1 (M+H)⁺; 38: m/z=214.3 (M+H)⁺; 39: m/z=200.1 (M+H)⁺; 40: m/z=213.1 (M+H)⁺; 41: m/z=217.1 (M+H)⁺; 42: m/z=243.1 (M+H)⁺; 43: m/z=215.1 (M+H)⁺.

### EXAMPLE 44

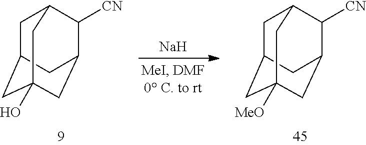

To a 177.0 mg of the nitrile 9 in 4.0 ml of DMF was added 90.2 mg of NaH (60% in mineral oil) in two portions at 0° C. and the resulting mixture was brought to rt and stirred for 1 h when 0.3 ml of MeI was added dropwise. The reaction continued for an additional hour when the reaction was quenched with NH₄Cl solution. The reaction mixture was extracted with EtOAc twice and the organic phases were combined, washed (by saturated NaCl solution), dried (over anhydrous Na₂SO₄), and concentrated to yield 143.2 mg nitrile 44. LC-MS (ESR): 44: m/z=192 (M+H)⁺.

### EXAMPLE 45

### Cell-Free Electrophysiology of M2 Proton Channels

The drug sensitivity of A/M2 proteins (wild type or S31N mutant) was measured using cell-free electrophysiology on solid supported membranes (SSM) (Schulz et al., Methods, 2008, 46, 97-103). For the SSM-based measurements cell membranes expressing the target protein are adsorbed to an SSM-coated gold sensor and the protein activity is evoked by substrate, or ligand concentration jumps, as appropriate. The resulting protein-dependent charge translocation is measured as a transient electrical current.

### SSM-Based Biosensors and Measurements

The biosensors were prepared with single-gold-electrode sensors from IonGate Biosciences (Germany) as described by the manufacturer. Briefly, the SSM was built on the gold electrode by applying first an alkane-thiol monolayer followed by a phospholipid monolayer on top of it. Subsequently, the SSM-coated sensors were covered with 100 μl of the ice cold M2 sensor preparation buffer (30 mM MES/KOH, pH 5.8, 140 KCl, 4 mM MgCl₂, 0.2 mM DTT) and incubated at 4° C. for 15 minutes. An aliquot of CHO membranes expressing M2 protein was rapidly thawed, diluted with the sensor preparation buffer to a final protein concentration of 0.5-1 μg/μL, and sonicated with a microsonicator by applying 5 bursts with an amplitude of 30% (ultrasonic processor UP 50 H with a MS 1 tip, Dr. Hielscher, Germany). 5-10 μg total protein of the sonicated membranes were loaded per each sensor, centrifuged for 30 min at 3,000 rpm and 4° C., and incubated for 24 hours at 4° C. The membrane-loaded biosensors were integrated into the fluidic system of the SURFE²R device (Surface Electrogenic Event Reader, IonGate Biosciences, Germany) and the A/M2 was activated through pH jumps by exchanging a “non-activating” solution (30 mM MOPS/KOH, pH 7.0, 140 KCl, 4 mM MgCl₂) for an “activating” solution (30 mM MES/KOH, pH 6.0, 140 KCl, 4 mM MgCl₂). For the inhibition experiments, the inhibitors were supplied at the same concentration to both solutions. Responses in the presence of the inhibitor (I) were normalized to the currents evoked by the application of the activating (pH 6.0) solution without inhibitor (Io) and are presented as % inhibition=100×(1−I/Io).

Representative compounds of Formula (I) were tested using the above protocol with results summarized in Table 1. The resulting inhibition is indicated as falling into one of three ranges: 51-95% (A), 11-50% (B), and 1-10% (C).

TABLE 1

| | | IonGate_M2(S31)_ | |
|---|---|---|---|
| | | Screen % | Reference to |
| Example | | Inhibition at 25 | Preceding Portion |
| No | Structure | μM¹ | of Disclosure |
| 1 | 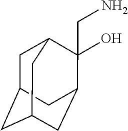 | A | Example 1 |
| 2 | 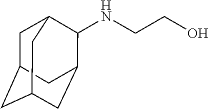 | A | Example 2 |
| 3 | 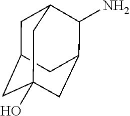 | B | Example 3 |
| 4 | 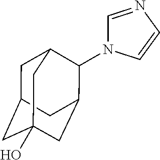 | B | Example 4 |
| 5 | 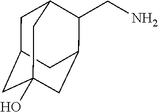 | B | Example 5 |
| 6 | 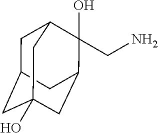 | B | Example 6 |
| 7 (Stereoisomer of 6) | 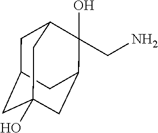 | B | Example 7 |
| 8 | 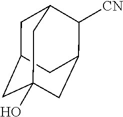 | B | Example 8 |
| 9 (Stereoisomer of 8) | 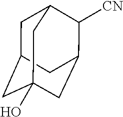 | C | Example 9 |
| 10 | 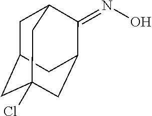 | C | Example 10 |
| 11 | 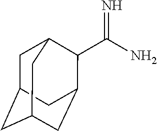 | A | Example 11 |
| 12 | 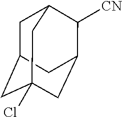 | B | Example 12 |
| 13 (Stereoisomer of 12) | 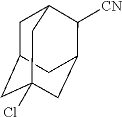 | B | Example 13 |
| 14 | 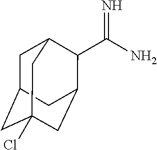 | A | Example 14 |
| 15 | 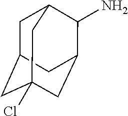 | A | Example 15 |
| 16 | 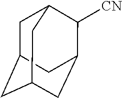 | A | Example 16 |
| 17 (Stereoisomer of 14) | 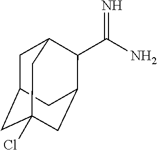 | A | Example 17 |
| 18 | 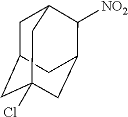 | C | Example 18 |
| 19 | 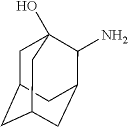 | A | Example 19 |
| 20 | 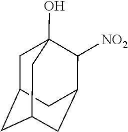 | B | Example 20 |
| 21 | 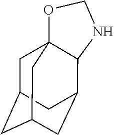 | A | Example 21 |
| 22 | 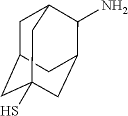 | A | Example 22 |
| 23 | 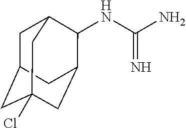 | B | Example 23 |
| 24 (Stereoisomer of 23) | 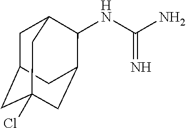 | A | Example 24 |
| 25 | 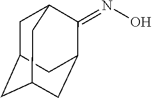 | B | Example 25 |
| 26 | 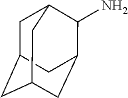 | B | Example 26 |
| 27 | 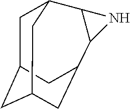 | B | Example 27 |
| 28 | 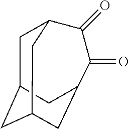 | B | Example 28 |
| 29 | 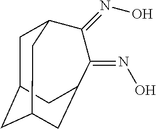 | B | Example 29 |
| 30 | 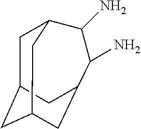 | A | Example 30 |
| 31 | 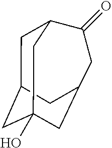 | B | Example 31 |
| 32 | 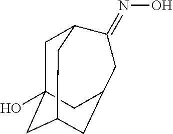 | B | Example 32 |
| 33 | 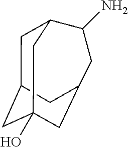 | B | Example 33 |
| 34 | 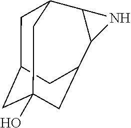 | B | Example 34 |
| 35 | 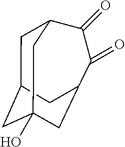 | C | Example 35 |
| 36 | 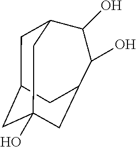 | C | Example 36 |
| 37 | 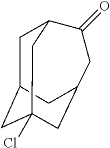 | B | Example 37 |
| 38 | 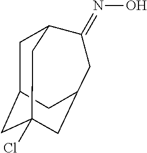 | B | Example 38 |
| 39 | 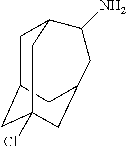 | B | Example 39 |
| 40 | 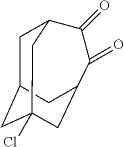 | C | Example 40 |
| 41 | 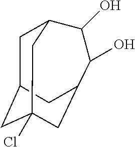 | C | Example 41 |
| 42 | 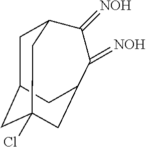 | B | Example 42 |
| 43 | 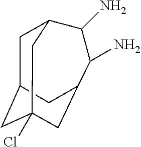 | C | Example 43 |
| 44 | 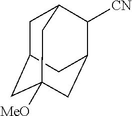 | C | Example 44 |

¹Activity range: 51-95% (A), 11-50% (B), and 1-10% (C)

In view of the preceding disclosure, it will be understood to those of ordinary skill in the art that the same can be performed within a wide and equivalent range of conditions, formulations, and other parameters without affecting the scope of the invention or any embodiment thereof.

What is claimed is:

1. A method for treating an influenza virus-affected disease state or infection comprising the step of administering to a subject in need thereof a composition comprising a compound of formula I: 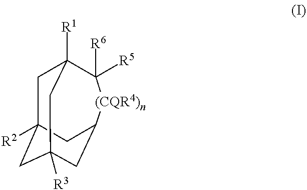
or a pharmaceutically acceptable salt thereof, wherein
R¹ is hydrogen, hydroxyl, halo, alkoxy, or together with R⁵ or R⁶ forms an optionally substituted five- or six-membered carbocyclic or heterocyclic ring;
R² and R³ are independently hydrogen, hydroxyl, halo, thiol, (C₁-C₃)alkylthio, or (C₁-C₃)alkoxy;
R⁴ is hydroxyl, oxo, oxime, amino, a three- to six-membered optionally substituted carbocyclic or heterocyclic ring, or together with R⁵ or R⁶ forms an aziridine group;
R⁵ and R⁶ are independently hydrogen, amino, nitro, cyano, hydroxyl, oxo, oximeamino(C₁-C₃)alkyl, hydroxy(C₁-C₃)alkylamino, formamidinyl, guanidinyl, oxime, —CH(X)(Y), a three- to six-membered heterocyclic ring, together with R⁴ forms an aziridine group, or together with R¹ forms an optionally substituted five- or six-membered carbocyclic or heterocyclic ring;
X and Y are independently hydrogen, hydroxyl, (C₁-C₃)alkyl, amino, amino(C₁-C₃)alkyl, (C₁-C₃)alkoxy, or (C₁-C₃)alkylamino;
Q is hydrogen or is absent; and,
n is 0 or 1;
wherein
if n=0, then at least one of R¹, R², R³, R⁵, and R⁶ is not hydrogen, and,
if R³ is hydrogen, then at least one of the following conditions apply:
n=1;
either R⁵ or R⁶ comprises moiety that includes at least one carbon atom and at least one nitrogen atom and is other than an amino-substituted cyclohexane or dithiane;
or, at least one of R⁵ and R⁶ is amino or nitro and R¹ is hydroxyl.

2. The method according to claim 1 wherein n=1

3. The method according to claim 1 wherein R³ is hydroxyl or thiol.

4. The method according to claim 3 wherein R¹ and R² are hydrogen.

5. The method according to claim 4 wherein R⁵ is hydrogen.

6. The method according to claim 5 wherein R⁶ is amino, —CH(X)(Y), cyano, hydroxyl, or oxo.

7. The method according to claim 6 wherein n=0.

8. The method according to claim 6 wherein n=1.

9. The method according to claim 8 wherein R⁴ is hydrogen, oxo, or hydroxyl.

10. The method according to claim 1 wherein R³ is halo.

11. The method according to claim 10 wherein R¹, R², R³, and R⁵ are hydrogen and n=0.

12. The method according to claim 1 wherein R³ is hydrogen.

13. The method according to claim 12 wherein R¹ is hydrogen.

14. The method according to claim 13 wherein n=1 and R² is hydrogen or hydroxyl.

15. The method according to claim 14 wherein R⁴ is amino, oxime, or oxo, and R⁵ is amino, oxime, or oxo.

16. The method according to claim 1 wherein said compound is: 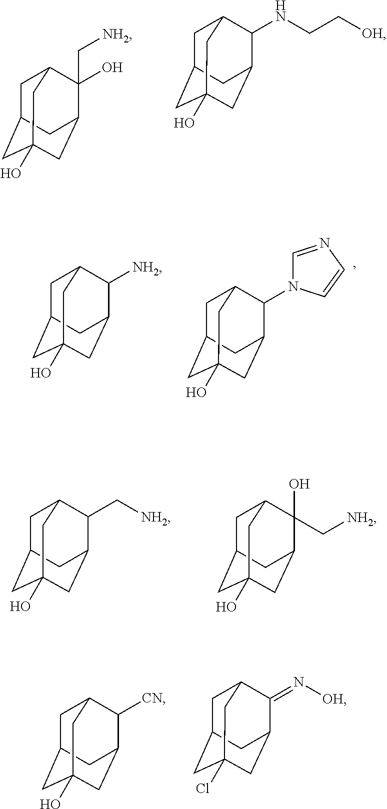 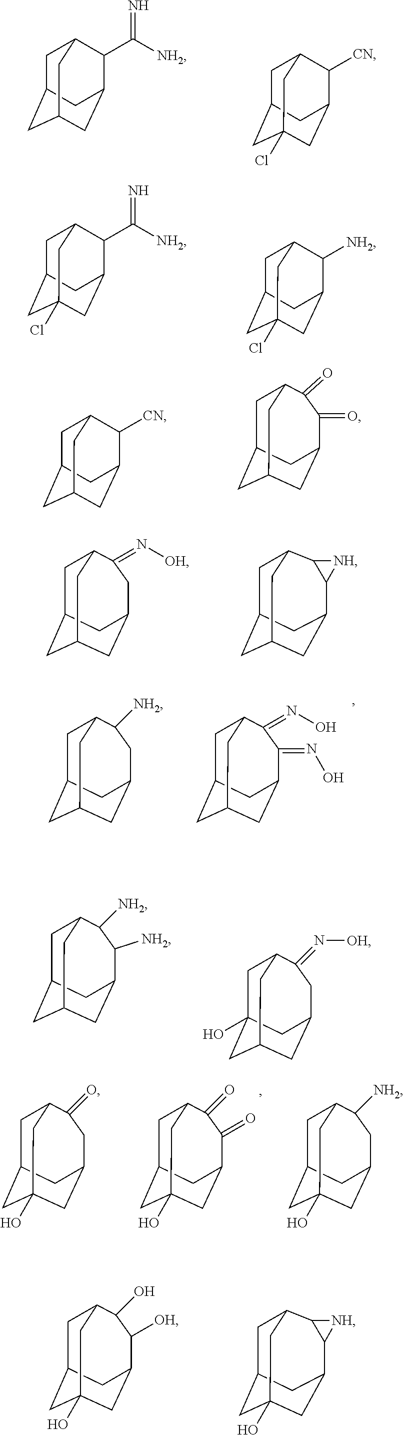 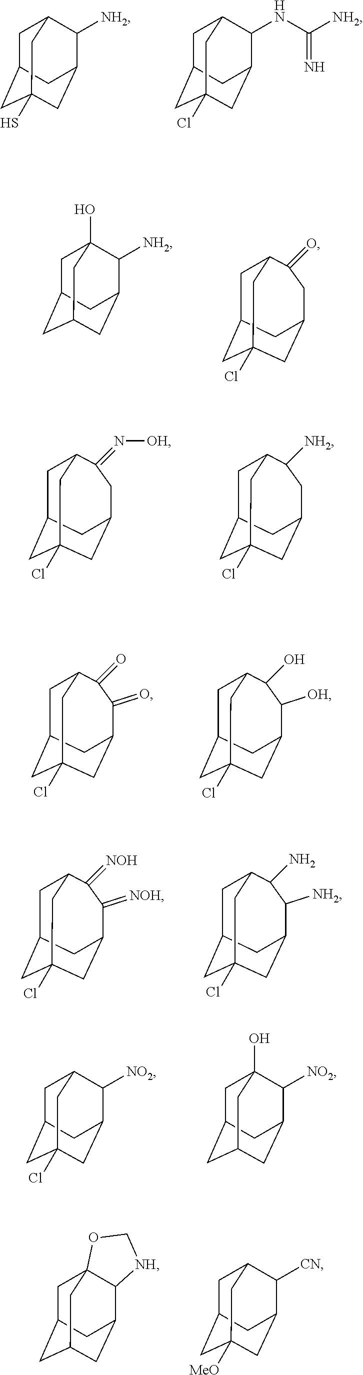
or a pharmaceutically acceptable salt or stereoisomer thereof.

17. A composition comprising a compound according to claim 1 or a pharmaceutically acceptable salt or stereoisomer thereof and a pharmaceutically acceptable carrier, diluent, or excipient.

18. The composition according to claim 17 further comprising a therapeutically effective amount of a further agent that modulates an influenza virus.

19. A compound according to formula II: 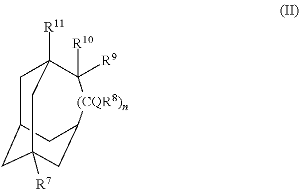
or a pharmaceutically acceptable salt thereof, wherein
n is 0 or 1;
R⁷ is hydroxyl or halo, or, if R¹¹ is hydroxyl or forms an optionally substituted five- or six-membered carbocyclic or heterocyclic ring together with R¹⁰, R⁷ may be hydrogen;
R⁸ is hydrogen, hydroxyl, amino, oxo, oxime, or together with R⁹ or R¹⁰ forms an aziridine group;
R⁹ is hydrogen, hydroxyl, cyano, amino, formamidinyl, guanidinyl, a three- to six-membered heterocyclic ring, or —CH(X)(Y);
if R⁷ is hydroxyl, then R¹⁰ is hydroxyl, cyano, formamidinyl, guanidinyl, a three- to six-membered heterocyclic ring, or —CH(X)(Y), or together with R⁸ forms an aziridine group, and if n is 1, R¹⁰ may additionally be amino;
if R⁷ is halo, then R¹⁰ is hydroxyl, nitro, formamidinyl, guanidinyl, a three- to six-membered heterocyclic ring, or —CH(X)(Y), and if n is 1, R¹⁰ may additionally be oxo, amino, oxime, or hydroxyl;
if R⁷ is hydrogen, then R¹⁰ is nitro, or together with R¹¹ forms an optionally substituted five- or six-membered carbocyclic or heterocyclic ring;
R¹¹ is hydrogen, hydroxyl, or together with R¹⁰ forms an optionally substituted five- or six-membered carbocyclic or heterocyclic ring;
X and Y are independently hydrogen, amino, amino(C₁-C₃)alkyl, or (C₁-C₃)alkylamino; and,
Q is hydrogen or is absent.

20. The compound according to claim 19 wherein n=0.

21. The compound according to claim 20 wherein R⁷ is hydroxyl.

22. The compound according to claim 21 wherein R⁹ is hydrogen or hydroxyl.

23. The compound according to claim 19 wherein n=1.

24. The compound according to claim 23 wherein R⁷ is hydroxyl.

25. The compound according to claim 24 wherein R⁹ is hydrogen and R¹⁰ is amino, hydroxyl, or, together with R⁸ forms an aziridine group.

26. The compound according to claim 19 wherein R⁷ is halo.

27. The compound according to claim 26 wherein R⁹ is hydrogen.

28. The compound according to claim 27 wherein R¹⁰ is oxo, amino, nitro, oxime, or hydroxyl.

29. The compound according to claim 28 wherein n=1 and R⁸ is hydrogen, oxo, hydroxyl, oxime, or amino.

30. The compound according to claim 19 wherein R⁷ is hydrogen.

31. The compound according to claim 19 wherein said compound is: 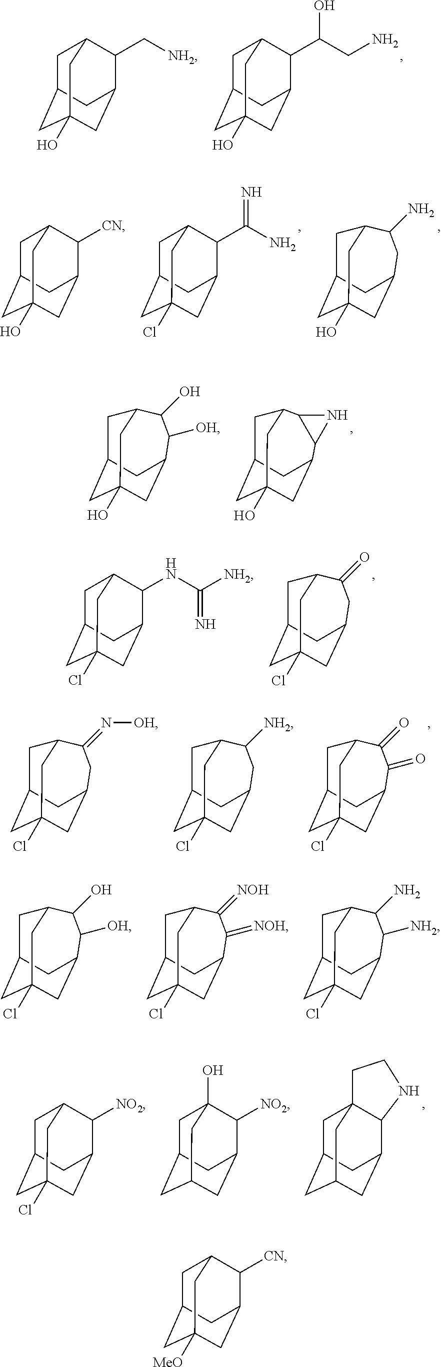
or a pharmaceutically acceptable salt or stereoisomer thereof.
